(19) United States
(12) Patent Application Publication
(10) Pub. No.: US 2009/0188964 A1
(43) Pub. Date: Jul. 30, 2009

(54) MEMBRANE AUGMENTATION, SUCH AS OF FOR TREATMENT OF CARDIAC VALVES, AND FASTENING DEVICES FOR MEMBRANE AUGMENTATION

(76) Inventor: Boris Orlov, Haifa (IL)

Correspondence Address: MARTIN D. MOYNIHAN d/b/a PRTSI, INC., P.O. BOX 16446, ARLINGTON, VA, 22215, US

(21) Appl. No.: 12/302,900

(22) PCT Filed: May 21, 2007
(86) PCT No.: PCT/IL07/00614

§ 371 Date: Dec. 1, 2008

Related U.S. Application Data
(60) Provisional application No. 60/860,805, filed on Nov. 24, 2006.
(60) Provisional application No. 60/832,162, filed on Jul. 21, 2006.
(60) Provisional application No. 60/814,572, filed on Jun. 19, 2006.
(60) Provisional application No. 60/809,848, filed on Jun. 1, 2006.
(60) Provisional application No. 60/832,142, filed on Jul. 21, 2006.

(51) Int. Cl. A61B 17/10

(52) U.S. Cl. 227/175.3; 227/176.1

(57) ABSTRACT

Disclosed are methods for augmenting tissue, for example membranes such as cardiac leaflets and tissue surrounding a cardiac valve, which in embodiments improve leaflet coaptation. Also disclosed are devices useful for augmenting tissue.

### RELATED APPLICATIONS

The present application gains benefit of the filing dates of U.S. patent application Nos. 60/809,848 filed 1 Jun. 2006; 60/814,572 filed 19 Jun. 2006; 60/832,142 filed 21 Jul. 2006; 60/832,162 filed 21 Jul. 2006 and 60/860,805 filed 24 Nov. 2006 all which are incorporated by reference as if fully set forth herein.

### FIELD AND BACKGROUND OF THE INVENTION

The present invention relates to the field of surgery and especially to surgical fastening devices such as surgical staplers useful, for example, in augmenting cardiac valve leaflets or in augmenting tissue surrounding a cardiac valve, for example to allow displacement of the intact cardiac valve. The use of surgical fastening devices of the present invention allows performance of otherwise lengthy and complex surgeries quickly and efficiently, for example in order to improve leaflet coaptation, for example in order to treat ischemic mitral regurgitation.

The human heart 10, depicted in cross sectional long axis view in FIG. 1, is a muscular organ that pumps deoxygenated blood through the lungs to oxygenate the blood and pumps oxygenated blood to the rest of the body by rhythmic contractions of four chambers.

After having circulated in the body, deoxygenated blood from the body enters the right atrium 12 through the vena cava 14. Right atrium 12 contracts, pumping the blood through a tricuspid valve 16 into the right ventricle 18. Right ventricle 18 contracts, pumping the blood through the pulmonary semi-lunar valve 20 into the pulmonary artery 22 which splits to two branches, one for each lung. The blood is oxygenated while passing through the lungs and reenters the heart to the left atrium 24.

Left atrium 24 contracts, pumping the oxygenated blood through the mitral valve 26 into the left ventricle 28. Left ventricle 28 contracts, pumping the oxygenated blood through the aortic semi-lunar valve 30 into the aorta 32. From aorta 32, the oxygenated blood is distributed to the rest of the body.

Physically separating left ventricle 28 and right ventricle 18 is interventricular septum 33. Physically separating left atrium 24 and right atrium 12 is an interatrial septum.

Mitral valve 26, depicted in FIG. 2A (top view) and in FIG. 2B (cross sectional long axis view) is defined by an approximately circular mitral annulus 34 that defines a mitral lumen 36. Attached to the periphery of mitral annulus 34 is an anterior leaflet 38 and a smaller posterior leaflet 40, leaflets 38 and 40 joined at commissures 41. Each leaflet is between about 0.8 and 2.4 mm thick and composed of three layers of soft tissue.

The typical area of mitral lumen 36 in a healthy adult is between 4 and 6 cm² while the typical total surface area of leaflets 38 and 40 is approximately 12 cm². Consequently and as depicted in FIG. 2B, leaflets 38 and 40 curve downwards into left ventricle 28 and coapt to accommodate the excess leaflet surface area, producing a coaptation surface 42 that constitutes a seal. The typical length of coaptation surface 42 in a healthy heart 10 of an adult is approximately 7-8 mm.

The bottom surface of anterior leaflet 38 and posterior leaflet 40 are connected to papillary muscles 44 at the bottom of left ventricle 28 by posterior chordae 46 and anterior chordae 48.

During diastole, left atrium 24 contracts to pump blood downwards into left ventricle 28 through mitral valve 26. The blood flows through mitral lumen 36 pushing leaflets 38 and 40 downwards into left ventricle 28 with little resistance.

During systole left ventricle 28 contracts to pump blood upwards into aorta 32 through aortic semi-lunar valve 30. Mitral annulus 34 contracts pushing leaflets 38 and 40 inwards and downwards, reducing the area of mitral lumen 36 by about 20% to 30% and increasing the length of coaptation surface 42. The pressure of blood in left ventricle 28 pushes against the bottom surfaces of leaflets 38 and 40, tightly pressing leaflets 38 and 40 together at coaptation surface 42 so that a tight leak-proof seal is formed. To prevent prolapse of leaflets 38 and 40 upwards into left atrium 24, papillary muscles 44 contract pulling the edges of leaflets 38 and 40 downwards through posterior chordae 46 and anterior chordae 48, respectively.

As is clear from the description above, an effective seal of mitral valve 26 is dependent on a sufficient degree of coaptation, in terms of length, area and continuity of coaptation surface 42. If coaptation surface 42 is insufficient or non-existent, there is mitral valve insufficiency, that is, regurgitation of blood from left ventricle 28 up into left atrium 24. A lack of sufficient coaptation may be caused by any number of physical anomalies that allow leaflet prolapse (e.g., elongated or ruptured chordae 46 and 48, weak papillary muscles 44) or prevent coaptation (e.g., short chordae 46 and 48, small leaflets 38 and 40).

Mitral valve insufficiency leads to many complications including arrhythmia, atrial fibrillation, cardiac palpitations, chest pain, congestive heart failure, fainting, fatigue, low cardiac output, orthopnea, paroxysmal nocturnal dyspnea, pulmonary edema, shortness of breath, and sudden death.

There are a number of pathologies that lead to a mitral valve insufficiency including collagen vascular disease, ischemic mitral regurgitation, myxomatous degeneration of leaflets 38 and 40 and rheumatic heart disease.

In ischemic mitral regurgitation (resulting, e.g., from myocardial infarction, chronic heart failure, or surgical or catheter revascularization), leaflets 38 and 40 and chordae 46 and 48 have normal structure and the mitral valve insufficiency results from altered geometry of left ventricle 28. As a result of ischemia, portions of the heart walls necrose. During healing, the necrotic tissue is replaced with unorganized tissue leading to remodeling of the heart which reduces coaptation through distortion of mitral annulus 34 and sagging of the outer wall of left ventricle 28 which displaces papillary muscles 44.

In FIGS. 3A (top view) and 3B (cross sectional long axis view), The reduction of coaptation resulting from ischemia is depicted for a mitral valve 26 of an ischemic heart 50 that has undergone mild remodeling and suffers from ischemic mitral regurgitation. In FIG. 3B is seen how an outer wall of left ventricle 28 sags outwards, displacing papillary muscles 44 downwards which, through chordae 46 and 48, pulls leaflets 38 and 40 downwards and apart, reducing coaptation. The incomplete closure of mitral valve 26 is seen in FIGS. 3A and 3B.

Initially, ischemic mitral regurgitation is a minor problem, typically leading only to shortness of breath during physical exercise due to the fact that a small fraction of blood pumped by left ventricle 28 is pumped into left atrium 24 and not through aortic semi-lunar valve 30, reducing heart capacity. To compensate for the reduced capacity, left ventricle 28 beats harder and consequently remodeling continues. Ultimately leaflet coaptation is entirely eliminated as leaflets 38 and 40 are pulled further and further apart, leading to more blood regurgitation, further increasing the load on left ventricle 28, and further remodeling. Ultimately, the left side of the heart fails and the person dies.

Apart from humans, mammals that suffer from mitral valve insufficiency include horses, cats, dogs, cows and pigs.

Currently, it is accepted to use open-heart surgical methods to improve mitral valve functioning by many different methods, including: modifying the subvalvular apparatus (e.g. lengthening the chordae) to improve leaflet coaptation; implanting an annuloplasty ring, e.g., as described in U.S. Pat. Nos. 3,656,185, 6,183,512 and 6,250,308 to force mitral valve annulus 34 into a normal shape; or implanting devices in the mitral valve to act as prosthetic leaflets, e.g., United States patent applications published as US 2002/065554, US 2003/0033009, US 2004/0138745 or US 2005/0038509.

Surgical augmentation of a mitral valve anterior leaflet 38 for improving mitral valve leaflet coaptation for treating ischemic mitral valve regurgitation is taught by Kincaid et al (Kincaid E H, Riley R D, Hines M H, Hammon J W and Kon N D in Ann. Thorac. Surg. 2004, 78, 564-568). An incision is made in the anterior leaflet almost from commissure to commissure. The edges of a roughly elliptical patch of material (e.g., bovine pericardium, 1 cm wide, 3 cm long) are sutured to either side of the incision augmenting the anterior leaflet by an amount roughly equal to the surface area of the patch. Additionally, a flexible annuloplasty ring is implanted to reshape the mitral annulus. Although effective, such augmentation is considered a complex surgical procedure performed only by cardiac surgeons having above average skill.

Open heart surgery of any kind is complex, requires long recovery time and is accompanied by a high rate of complications and death. The failure rate of the mitral valve function improvement operations is very high both due to objective reasons relating to patient condition and the subjective difficulty of performing such operations repeatedly correctly. Even when successfully performed, persons having undergone such surgeries have an increased chance of infection and stroke, and are often required to use anticoagulant agents for the rest of their lives. Often there is a need to repeat the surgery after a few years.

Any significant reduction of time in an open heart surgery increases the chance of success of the operation. Further, any way in which a heart surgery may be performed more accurately will generally provide a consistently better prognosis.

It would be highly advantageous to have a quick and simple method for performing open heart operations such as augmenting cardiac valve leaflets or otherwise improving leaflet coaptation, to reduce mitral insufficiency, for example for treating subjects suffering from ischemic mitral valve regurgitation.

### SUMMARY OF THE INVENTION

The present invention successfully addresses at least some of the shortcomings of the prior art by providing methods of tissue augmentation which in embodiments improves cardiac valve leaflet coaptation, which may be useful in treating conditions, for example mitral insufficiency such as ischemic mitral regurgitation. The present invention also provides surgical fastening devices such as surgical staplers and related components that allow procedures such as leaflet augmentation or cardiac valve displacement to be performed quickly, accurately and of consistent quality with less dependence on the skill level or degree of exhaustion of the performing surgeon.

In an embodiment of the present invention, augmentation of a membrane (increase of the surface area of the membrane) such as a cardiac valve leaflet (in a manner reminiscent to the taught by Kincaid et al) is performed more quickly and more easily than known in the art.

Thus, according to the teachings of the present invention there is provided a method of augmenting a membrane, comprising: a) slitting an in vivo membrane so as to produce a slit having a first slit edge and a second slit edge; b) providing a patch of tissue having a periphery as a membrane augmenting implant; c) placing the patch of tissue to define an overlap region comprising an overlap of a first portion of the periphery with the first slit edge and an overlap of a second portion of the periphery with the second slit edge; and d) securing the patch to the membrane around a plurality of locations of the overlap region substantially simultaneously, thereby augmenting the membrane by increasing a surface area of the membrane with the patch.

In embodiments, the in vivo membrane augmented is a leaflet of a cardiac valve. In embodiments, the cardiac valve is a mitral valve. In embodiments, the leaflet is a posterior mitral valve leaflet. In embodiments, the augmenting of a leaflet of a cardiac valve improves coaptation of the leaflets of the mitral valve.

In embodiments, the slit has a shape selected from the group consisting of straight, curved and wavy.

In embodiments, the slitting of the membrane and the placing of the patch is substantially simultaneous.

In embodiments, the slitting of the membrane, the placing of the patch and the securing of the patch to the membrane is substantially simultaneous.

In embodiments, the overlap region is substantially a closed curve circumscribing the slit. Typical suitable shapes of the overlap region include but are not limited to circles, ovals, ellipses, oblate ovals, oblate ellipses and oblate circles.

In embodiments, during the securing of the patch to the membrane, the area of the membrane circumscribed by the overlap region is less than the surface area of the patch circumscribed by the first portion and by the second portion of the patch.

In embodiments, the securing of the patch to the membrane comprises: i) piercing the patch and the membrane at the plurality of locations to make a plurality of holes in the overlap region substantially simultaneously; ii) passing lockable fasteners through the holes; and iii) locking the lockable fasteners so as to secure the patch to the first slit edge and the second slit edge through the overlap region.

In embodiments, the piercing of the patch and the membrane as well as the passing of the lockable fasteners through the holes is substantially simultaneously performed by driving a piercing element of the lockable fasteners (e.g., the legs of a staple) through the patch and the membrane.

In embodiments, the slitting of the membrane, the placing of the patch and the piercing of the patch and the membrane is substantially simultaneous.

In embodiments, locking a lockable fastener comprises deforming the lockable fastener, for example clinching a staple.

In embodiments, locking a lockable fastener comprises securing a locking component to the lockable fastener.

In embodiments, during the piercing of the patch and the membrane, the area of the membrane circumscribed by the holes through the first slit edge and the second slit edge is less than the surface area of the patch circumscribed by the holes through the periphery of the patch. In embodiments, during the piercing, the patch is buckled.

According to the aspect of the present invention, the tissue surrounding a cardiac valve is augmented (e.g., the surface area of tissue between the valve annulus and the valve itself is increased) more quickly and more easily than known in the art. In embodiments, such augmenting allows the cardiac valve to be displaced, which in embodiments increases coaptation of the valve leaflets.

Thus according to the teachings of the present invention there is also provided a method of augmenting the tissue surrounding a cardiac valve, comprising: a) excising leaflets of a cardiac valve with an incision having a shape of a closed curve, so as to define a valve seat edge of the incision and a valve periphery edge of the incision; b) providing an implant including a wall, the wall delimited by two edges each in the shape of a closed curve and defining a lumen. (e.g., a tube or annulus) as a cardiac valve augmenting implant; c) placing the implant to define a proximal overlap region comprising an overlap of a first portion near a first edge of the implant with the valve seat edge d) placing the implant to define a distal overlap region comprising an overlap of a second portion near a second edge of the implant with the valve periphery edge; e) securing the first portion of the implant to the valve seat edge around a plurality of locations of the proximal overlap region; and f) securing the second portion of the implant to the valve periphery edge around a plurality of locations of the distal overlap region, substantially simultaneously, thereby augmenting a surface area of tissue surrounding the cardiac valve with the implant, and in embodiments allowing displacement of the cardiac valve. In embodiments, spare portions of the implant are trimmed. It is important to note that the steps of the method may be performed in any rational order and not necessarily in the order listed above. For example, in embodiments, a precedes c and/or d and/or e and/or f; a succeeds c and/or d and/or e and/or f; c precedes d and/or f, C precedes d and/or f, d precedes c and/or e; f precedes c and/or e. It is important to note, as discussed below, in preferred embodiments one or more steps are substantially simultaneous.

In embodiments, the cardiac valve is a mitral valve. In embodiments, the augmentation of the tissue surrounding the valve improves coaptation of leaflets of the cardiac valve.

In embodiments, the incision is of a shape selected from the group consisting of circle, oval, ellipse, oblate oval, oblate ellipse and oblate circle.

In embodiments, securing the first portion of the implant to the valve seat edge around a plurality of locations of the proximal overlap region is performed substantially simultaneous for the plurality of location.

In embodiments, excising the leaflets, placing the implant to define the proximal overlap zone and securing the first portion of the implant to the valve seat edge are substantially simultaneous.

In embodiments, excising the leaflets, placing the implant to define the distal overlap zone, and securing the second portion of the implant to the valve periphery edge are substantially simultaneous.

In embodiments, excising the leaflets, placing the implant to define the proximal overlap zone, placing the implant to define the distal overlap zone, securing the first portion of the implant to the valve seat edge and securing the second portion of the implant to the valve periphery edge are substantially simultaneous.

In embodiments, the proximal overlap region is substantially a closed curve circumscribing the incision. Typical shapes of closed curves include, but are not limited to circles, ovals, ellipses, oblate ovals, oblate ellipses and oblate circles.

In embodiments, the distal overlap region is substantially a closed curve surrounded the incision. Typical shapes of closed curves include, but are not limited to circles, ovals, ellipses, oblate ovals, oblate ellipses and oblate circles.

In embodiments, the surface area of the walls of the implant between the proximal overlap region and the distal overlap region is greater than the surface area of the tissue between the proximal overlap region and the distal overlap region. In embodiments, during the placing of the implant, the wall of the implant is buckled.

In embodiments, securing the first portion of the implant to the valve seat edge comprises: i) piercing the implant and the tissue surrounding the valve seat edge at the plurality of locations to make a plurality of holes in the proximal overlap region substantially simultaneously; ii) passing lockable fasteners through the holes; and iii) locking the lockable fasteners so as to secure the implant to the valve seat edge through the proximal overlap region.

In embodiments, securing the second portion of the implant to the valve periphery edge comprises: i) piercing the implant and tissue surrounding the valve periphery edge at the plurality of locations to make a plurality of holes in the distal overlap region substantially simultaneously; ii) passing lockable fasteners through the holes; and iii) locking the lockable fasteners so as to secure the implant to the valve periphery edge through the distal overlap region.

In embodiments, piercing the implant and the tissue and passing the lockable fasteners through the holes is substantially simultaneously performed by driving a piercing element of the lockable fasteners through the implant and the tissue.

In embodiments, locking a lockable fastener comprises deforming the lockable fastener, for example clinching a staple.

In embodiments, locking a lockable fastener comprises securing a locking component to the lockable fastener.

As noted above, an implant used in augmenting the tissue surrounding a cardiac valve in accordance with the teachings of the present invention includes a wall, the wall delimited by two edges each in the shape of a closed curve and defining a lumen. Suitable closed curve shapes of the edges of an implant include, but are not limited to circles, ovals, ellipses, oblate ovals, oblate ellipses and oblate circles. Any suitable material or combination of materials may be used for fashioning a wall of an implant, both synthetic and biological as is detailed hereinbelow.

In embodiments, an implant is substantially a flat sheet of material with a hole therethrough, where the first edge is the outer edge of the flat sheet and the second edge is the edge of the hole. In such embodiments, the first region, that which is secured to the valve seat edge of the incision is a portion of the sheet closer to the first edge (edge of the sheet) than the second region which is closer to the second edge (the edge of the hole) and to which the valve periphery edge of the incision is secured. In embodiments, the flat sheet of material is in the shape of an annulus or ring. In embodiments the two edges are of the same shape. In embodiments, the two edges describe shapes that are substantially concentric.

In embodiments, an implant is substantially a tube of material having a proximal end and a distal end with a lumen passing therebetween, where the first edge is the rim of the proximal end and the second edge is the rim of the distal end. In such embodiments, the first region, that which is secured to the valve seat edge of the incision is a portion of the tube closer to the first edge (proximal rim) than the second region which is closer to the second edge (distal rim) and to which the valve periphery edge of the incision is secured. In embodiments, the tube is substantially parallel walled. In embodiments, the distal rim and the proximal rim are of substantially the same size. In embodiments, the distal end and the proximal end are coaxial. In embodiments, the distal end and the proximal end are not-coaxial. In embodiments, the proximal rim is substantially larger than the distal rim. In embodiments, the tubular wall is substantially a truncated cone. In embodiments, the distal end and the proximal end are coaxial. In embodiments, the distal end and the proximal end are not-coaxial. In embodiments, the tubular wall is substantially frustoconical. In embodiments, the ends of the truncated cone are substantially not parallel.

Embodiments of the methods of the present invention may be performed quickly and/or efficiently and/or more accurately with embodiments of fastening devices of the present invention.

According to the teachings of the present invention there is also provided a surgical fastening device useful for securing a cardiac valve and an implant together, comprising: a) a device body comprising a fastener magazine having a fastener ejection ring (in embodiments, the fastener ejection ring having the shape of a closed curve) and configured to project a plurality of tissue-piercing elements of one or more lockable fasteners (preferably, substantially in parallel to an axis) in the shape of a closed curve from the fastener magazine; and b) an anvil configured to act as a counterpoise during projection of the tissue-piercing elements which comprises an anvil head including an anvil head top surface having a shape of a closed-curve and a size substantially similar to that of the fastener ejection ring, wherein the anvil is securable to the device body through a stem surrounded by the fastener ejection ring, so that when secured, the anvil head surface is opposite the fastener ejection ring, wherein the anvil head comprises at least two mateable anvil head parts, wherein each mateable anvil head part spans a sector less than 360° of the closed curve of the anvil head. Typical lockable fasteners include, but are not limited to, staples, cotter pins, studs, rivets, and two-part rivets. In embodiments, the device is a stapler and the one or more lockable fasteners are staples.

Such a fastening device is useful for associating, for example, a mitral valve edge with an edge of an implant as described above. Generally, the anvil head parts are passed through the mitral valve annulus and around the chordae, and mated to constitute a whole anvil head on the distal (ventricular) side of the mitral valve with the stem passing through the mitral valve lumen. When activated, the fastening device drives a plurality of lockable fastener substantially simultaneously through the edge of the implant and the mitral valve edge. The anvil is then taken apart and the parts withdrawn through the mitral valve lumen, while avoiding the chordae.

Thus, according to the teachings of the present invention, there is also provided an anvil for a surgical fastening device useful for securing a cardiac valve to an implant, comprising at least two mateable anvil head parts that when mated the anvil head parts constitute an anvil head that is substantially a closed curve including an anvil head top surface, wherein each the mateable anvil head part spans a sector less than 360° of the closed curve.

In embodiments, the closed curve shape of the anvil and which the fasteners are projected is of a shape selected from the group consisting of circle, oval, ellipse, oblate oval, oblate ellipse and oblate circle. It is understood that embodiments comprise also anvil heads that are not entirely closed curves but also substantially closed curves. For example, in embodiments the anvil head describes a split curve that constitute 350° of a closed curve where the 10° gap does not significantly adversely effect the functioning of the device. For example, in embodiments the anvil head is intermittent with a plurality of small gaps that do not significantly adversely effect the functioning of the device.

In embodiments, the anvil is of 2, 3, 4, 5, 6 or even more anvil head parts. That said, in a preferred embodiment, the anvil comprises two mateable anvil head parts, a first anvil head part and a second anvil head part, which when mated together spanning the entire circumference of the closed curve. In embodiments, the first anvil head part spans a sector of between about 120° and about 240° of the closed curve. In embodiments, the first anvil head part spans a sector of between about 160° and about 200° of the closed curve.

In embodiments, each anvil head part comprises an inwardly extending crosspiece. Such a crosspiece is generally relatively narrow, spanning no greater than about 45°, no greater than about 30°, no greater than about 15° and even no greater than about 10°.

In embodiments, an anvil comprises a stem attached to at least one the anvil head part through a crosspiece, the crosspiece inwardly extending from the anvil head part to the stem. In embodiments, the stem attached to only one the anvil head part. In embodiments, the stem is split into a plurality of mateable parts, the parts attached to different anvil head parts, and configured so that when the anvil head parts are properly mated the stem parts are properly mated.

In embodiments, the top surface of the anvil comprises a tray for presenting locking components to engage lockable fasteners projected from the surgical fastening device, e.g., the locking components of two-part rivets.

In embodiments, the top surface of the anvil comprises features for facilitating deformation of lockable fasteners projected from the surgical fastening device, for example for a stapler typical deformation facilitation, deformation facilitating features comprise staple clinching grooves.

According to the teachings of the present invention there is also provided for a surgical fastening device useful for augmenting tissue (for example in accordance with the methods of the present invention), comprising: a) a device body including a fastener magazine functionally associated with a fastener ejection ring (in embodiments, in the shape of a closed curve) at a distal end of the device body and configured, upon triggering, to project a plurality of tissue-piercing elements of one or more lockable fasteners from the fastener magazine through the fastener ejection ring in a direction (preferably, substantially in parallel to an axis) in the shape of the closed curve; b) an anvil configured to act as a counterpoise during the projection of the tissue-piercing elements which comprises an anvil head including an anvil head top surface having a shape substantially of a closed curve and a size substantially similar to that of the fastener ejection ring, the anvil secured to the device body through a stem, so that when secured, the anvil head surface is opposite the fastener ejection ring; c) an implant container at the distal end of the device body including an outwardly directed opening (e.g., directed substantially in the direction substantially in parallel to the axis) surrounded by the fastener ejection ring; and d) a knife driver defining a part of a wall of the implant container configured, upon triggering, to push an object held in the implant container out through the opening. As is explained below, such a device is useful, for example, in implementing the methods of the present invention efficiently, in embodiments by allowing cutting of tissue and fastening an implant with one action.

In embodiments, the closed curve shape of the anvil and which the lockable fasteners are projected is of a shape selected from the group consisting of circle, oval, ellipse, oblate oval, oblate ellipse and oblate circle. It is understood that embodiments comprise also anvil heads that are not entirely closed curves but also substantially closed curves, as discussed above.

Typical lockable fasteners include, but are not limited to, staples, cotter pins, studs, rivets, and two-part rivets. In embodiments, the device is a stapler and the one or more lockable fasteners are staples and, preferably, the anvil head top surface including a staple clinching groove ring opposite the fastener ejection ring when the anvil is secured to the device body.

In embodiments, the anvil head is reversibly detachable from the device body.

In embodiments, the device further comprises a trigger to trigger the projection of the tissue-piercing elements of the lockable fasteners contained in the fastener magazine and to trigger the pushing of an object held in the implant container by the knife driver. In embodiments, the trigger is configured to trigger the projection and the pushing simultaneously.

In embodiments, the device further comprises a cutting blade, physically separate from the device body and configured to be accommodated within the implant container together with an implant.

In embodiments, the anvil head top surface including a cutting blade accepting slot opposite the opening of the implant container when the anvil is secured to the device body, configured to accept the cutting blade.

In embodiments, the cutting blade is configured to make a single slit, for example having a shape selected from the group consisting of straight, curved and wavy. In embodiments, such a cutting blade allows implementation of the method of membrane augmentation in accordance with the teachings of the present invention. In embodiments, the anvil head top surface including a cutting blade accepting slot opposite the opening of the implant container when the anvil is secured to the device body, configured to accept the cutting blade.

In embodiments, the fastener magazine of the device is an outer fastener magazine, the fastener ejection ring is an outer fastener ejection ring, the device further comprises: e) a second fastener magazine functionally associated with an inner fastener ejection ring at a the distal end of the device body in the shape of a closed curve; wherein the implant container is in the shape of a closed curve surrounding the second fastener ejection ring; wherein the stem of the anvil is surrounded by the second fastener ejection ring; and where, the device body is further configured, upon triggering, to project a plurality of tissue-piercing elements of one or more fasteners from the inner fastener magazine in the shape of the closed curve through the inner fastener ejection ring in the direction. In embodiments, the anvil head comprises at least two mateable anvil head parts, wherein each the mateable anvil head part spans a sector of the closed curve of the anvil head, in embodiments substantially as described above. In embodiments, the device further comprises a cutting blade physically separate from the device body and configured to be accommodated within the implant container together with an implant. In embodiments, the cutting blade is configured to make an incision having a closed curved shape, including but not limited to a shape selected from the group consisting of circle, oval, ellipse, oblate oval, oblate ellipse and oblate circle. In embodiments, the cutting blade is a linear strip of sufficient flexibility to be bent into the closed curve shape by the implant container. In embodiments, the cutting blade is a loop of sufficient flexibility to be collapsed to an elongated shape.

In embodiments of a device of the present invention, a device body is configured to accept a cartridge which includes fasteners, an implant and/or a cutting blade. For use, a cartridge is attached to the device body.

Thus, according to the teachings of the present invention there is also provided a cartridge for a surgical fastening device, comprising: a) a cartridge body with a distal end and a proximal end, configured for association with a surgical fastening device through the proximal end of the cartridge body; b) a fastener magazine functionally associated with a fastener ejection ring at the distal end of the cartridge body (preferably in the shape of a closed curve); c) a plurality of tissue-piercing elements of one or more lockable fasteners held in the fastener ejection magazine and directed towards the fastener ejection ring so as to describe a closed curve; d) an implant container at the distal end including an outwardly directed opening surrounded by the fastener ejection ring; e) contained within the implant container, an implant comprising a patch of tissue having a periphery wherein the periphery extends beyond the fastener ejection ring; and f) a cutting blade contained within the implant container and nestled in the implant with a cutting edge directed towards the opening of the implant container.

Typical lockable fasteners include, but are not limited to, staples, cotter pins, studs, rivets, and two-part rivets. In embodiments, the device is a stapler and the one or more lockable fasteners are staples.

In embodiments, the cutting blade is configured to make a single slit, for example having a shape selected from the group consisting of straight, curved and wavy. In embodiments, the cutting blade comprises a strip of a suitable material, e.g., stainless steel, titanium, titanium alloys and the like.

According to the teachings of the present invention there is also provided a cartridge for a surgical fastening device, comprising: a) a cartridge body with a distal end and a proximal end, configured for association with a surgical fastening device through the proximal end of the cartridge body; b) an outer fastener magazine functionally associated with an outer fastener ejection ring at the distal end of the cartridge body (preferably in the shape of a closed curve); c) a plurality of tissue-piercing elements of one or more lockable fasteners held in the outer fastener ejection magazine and directed towards the outer fastener ejection ring so as to describe a closed curve; d) an implant container in the shape of a closed curve at the distal end including an outwardly directed opening surrounded by the fastener ejection ring; e) an inner fastener magazine functionally associated with an inner fastener ejection ring at the distal end of the cartridge body (preferably in the shape of a closed curve) surrounded by the implant container; f) a plurality of tissue-piercing elements of one or more lockable fasteners held in the inner fastener ejection magazine and directed towards the inner fastener ejection ring so as to describe a closed curve; g) contained within the implant container, an implant including a wall, the wall delimited by two edges each in the shape of a closed curve, and defining a lumen, wherein a first of the two edges extends outwards beyond the outer fastener ejection ring and a second of the two edges extends inwards beyond the inner fastener ejection ring; and h) a cutting blade contained within the implant container and nestled in the implant with a cutting edge directed towards the opening of the implant container.

Typical lockable fasteners include, but are not limited to, staples, cotter pins, studs, rivets, and two-part rivets. In embodiments, the device is a stapler and the one or more lockable fasteners are staples.

In embodiments, the cutting blade is configured to make an incision having a closed curve shape single slit, for example having a shape selected from the group consisting of circle, oval, ellipse, oblate oval, oblate ellipse and oblate circle, In embodiments, the cutting blade is a strip of sufficient flexibility to be bent into the implant container to have the desired closed curve shape. In embodiments, the cutting blade is a loop of sufficient flexibility to be collapsed to an elongated shape, as discussed above. The cutting blade comprises a suitable material, e.g., stainless steel, titanium, titanium alloys and the like.

As noted above and detailed below, lockable fasteners are used to implement embodiments of the present invention. Examples of lockable fasteners useful for implementing the teachings of the present invention that are lockable by deformation include, but are not limited to, staples, cotter pins, studs and rivets.

Examples of lockable fasteners useful for implementing the teachings of the present invention that are lockable by engagement with a discrete locking component (e.g., unidirectional locking rings) include, but are not limited to, two-part rivets.

According to the teachings of the present invention there is also provided a lockable fastener useful for securing the periphery of a cardiac valve to an implant material (for example, in accordance with a method of the present invention), comprising: a) a loop-shaped body with a bottom side defining a lumen and describing at least a 320° sector about an axis; and b) at least three elongated piercing elements protruding from the bottom side substantially parallel to the axis. In embodiments, the loop-shaped body describes at least a 340° sector about the axis and even a complete loop about the axis. In embodiments, the piercing elements are configured to deform so that the fastener is lockable by deformation of the piercing elements, preferably by bending, preferably in a direction tangential to the body, upon sufficiently forceful contact with an anvil when projected from an appropriately configured surgical fastening device. In embodiments, the fastener is lockable by securing at least one discrete locking component to the piercing elements (which are configured therefore). In embodiments, a set of at least two lockable fasteners such as above is provided, where the two fasteners are coaxially nestable, so that when coaxially nested, a coaxial gap between the outer edge of the body of the smaller fastener and the inner edge of the body of the larger fastener is at least 0.1 mm and preferably less than about 10 mm. Such a gap is configured to accommodate an implant and a cutting blade, as described above.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

As used herein, the indefinite articles “a” and “an” mean “at least one” or “one or more”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 (prior art) is a schematic depiction of a healthy heart in cross section;

FIGS. 2A and 2B (prior art) depict a mitral valve of a healthy heart;

FIGS. 3A and 3B (prior art) depict a mitral valve of a heart suffering from ischemic mitral regurgitation related to incomplete coaptation of the leaflets of the mitral valve;

FIGS. 4A-4I depict a membrane augmenting stapler of the present invention and a use thereof for augmenting a mitral anterior leaflet;

FIG. 5A depicts cardiac valve leaflet augmentation in accordance with the teachings of copending PCT patent application identified by Attorney Docket No. 39351 using an annuloplasty ring with an associated membrane;

FIGS. 5B-5C depict mitral valve displacement in accordance with the teachings of copending PCT patent application identified by Attorney Docket No. 39351 using an annuloplasty ring with an associated membrane;

FIG. 5D depicts mitral valve displacement in accordance with the teachings of copending PCT patent application identified by Attorney Docket No. 39351 using a substantially tubular implant;

FIGS. 6A and 6B depict an embodiment of a cardiac valve periphery stapler including a multipart anvil comprising two anvil parts;

FIGS. 6C-6F depict an embodiment of a method of displacing a mitral valve with the help of a tubular implant where the mitral valve is secured to the implant with the help of the cardiac valve periphery stapler depicted in FIGS. 6A and 6B.

FIG. 7 depicts a multipart anvil of an embodiment of a cardiac valve periphery stapler comprising two anvil head parts neither of which includes a substantial portion of an anvil stem;

FIG. 8A depicts an embodiment of a valve displacement stapler of the present invention;

FIG. 8B depicts an embodiment of a cutting blade for use with a stapler of FIG. 8A;

FIG. 8C depicts a frustoconical implant useful as an implant used in conjunction with a stapler of FIG. 8A;

FIGS. 8D-8J depict an embodiment of a method of displacing a mitral valve with the help of a tubular implant where the mitral valve is secured to the implant with the help of the valve displacement stapler depicted in FIGS. 8A and 8B; and

FIGS. 9A-9G depicts fasteners useful in implementing the teachings of the present invention.

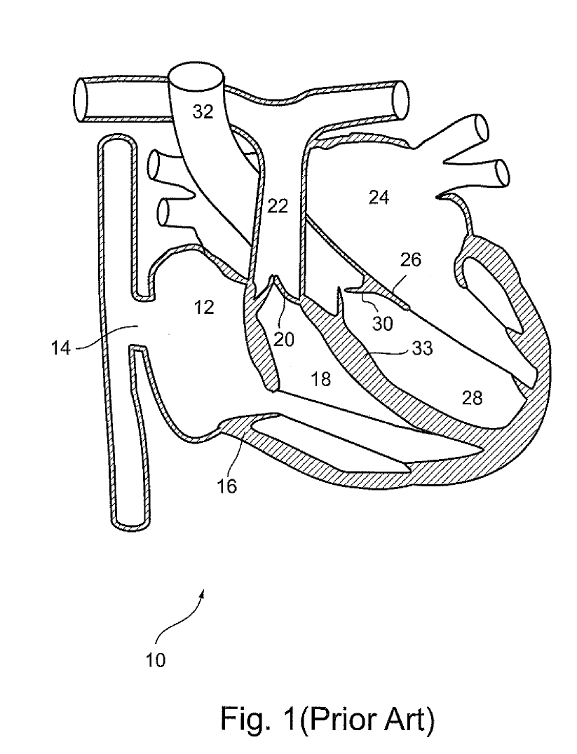

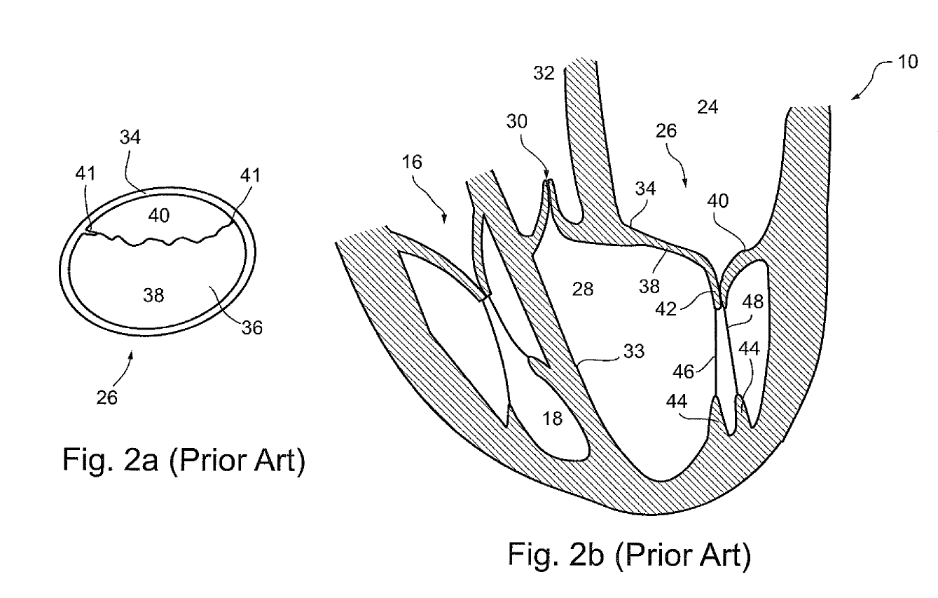

### DESCRIPTION OF EMBODIMENTS

The present invention relates to methods and devices for tissue augmentation that in embodiments are useful for improving cardiac leaflet coaptation, especially of the mitral valve. Generally, according to the teachings of the present invention the subvalvular apparatus is preserved.

Embodiments of the present invention successfully address at least some of the shortcomings of the prior art by providing a simple method of augmenting membranes such as cardiac valve leaflets. Thus, the teachings of the present invention allow a cardiac leaflet to be augmented and therefore embodiments are useful for treating a condition where cardiac valve augmentation is beneficial, such as mitral valve insufficiency, for example ischemic mitral regurgitation.

Embodiments of the present invention successfully address at least some of the shortcomings of the prior art by providing a simple method of augmenting the tissue around a cardiac valve. In embodiments, this leads to cardiac valve displacement that improves leaflet coaptation and may be useful for treating ischemic mitral valve regurgitation. Thus, the teachings of the present invention allow a cardiac valve to be displaced and therefore embodiments are useful for treating a condition where cardiac valve displacement is beneficial, such as mitral valve insufficiency, for example ischemic mitral regurgitation.

By treating a condition is meant curing the condition, treating the condition, preventing the condition, treating symptoms of the condition, curing symptoms of the condition, ameliorating symptoms of the condition, treating effects of the condition, ameliorating effects of the condition, and preventing results of the condition.

As is understood from the above, embodiments of the present invention are useful in augmenting membranes, especially cardiac valve leaflets, especially for treating a condition where cardiac valve augmentation is beneficial and are also useful in displacing a cardiac valve especially for are useful for treating a condition where cardiac valve displacement is beneficial. In order to simplify understanding the teachings of the present invention embodiments of the present invention will be discussed in the context of treating a mitral valve suffering from ischemic mitral regurgitation where the teachings of the present invention are directed to increasing leaflet coaptation and thus treat the ischemic mitral regurgitation.

Embodiments of the present invention relate to surgical fastening devices that project lockable fasteners such as staplers and components thereof useful in performing tissue augmentation and quickly and efficiently. In order to simplify understanding the teachings of the present invention embodiments of the present invention will be discussed with reference to surgical staplers, fastening devices that implant staples in the body in order to fasten or secure tissue. As is discussed below, in embodiments surgical fastening devices that deploy fasteners other than staplers are also useful in implementing the teachings of the present invention.

Staples are lockable fasteners well-known in the art. A typical staple consists of a length of wire or the like bent so as to describe a square U-shape, having two parallel legs and a perpendicular connecting crown, where the tips of the two legs constitute piercing elements.

A stapler is a device configured to drive and clinch one or more staples simultaneously through at least two layers of membrane in order to secure the layers together. The staples are held inside a staple magazine in a stapler body with the piercing elements pointing outwards of a staple ejection slot. Positioned across from the ejection slot at some distance is an anvil provided with clinching grooves across from each piercing element to act as a counterpoise.

For use, layers to be secured are pinched between the staple ejection slot and the anvil so that the piercing elements are perpendicular to the surface of the layers. When the stapler is activated, a drive blade presses downwards on the staple crown applying a force parallel to the axis of the piercing elements, driving the piercing elements down through the layers until the piercing elements clear the last of the layers and enter the clinching grooves. As the drive blade presses downwards further, the piercing elements and the legs are forced to curve inwards so that the staple is bent into a loop shape where the layers are clamped between the curved legs and the staple crown, locking the staple

Surgical staplers are well known in the art, see for example, Examples of prior art surgical staplers of the type described can be found in U.S. Pat. Nos. 2,853,074, 3,874,384, 3,079,608, 3,225,996, 3,489,330, 3,604,561 and 3,873,016.

Membrane Augmentation such as Cardiac Leaflet Augmentation

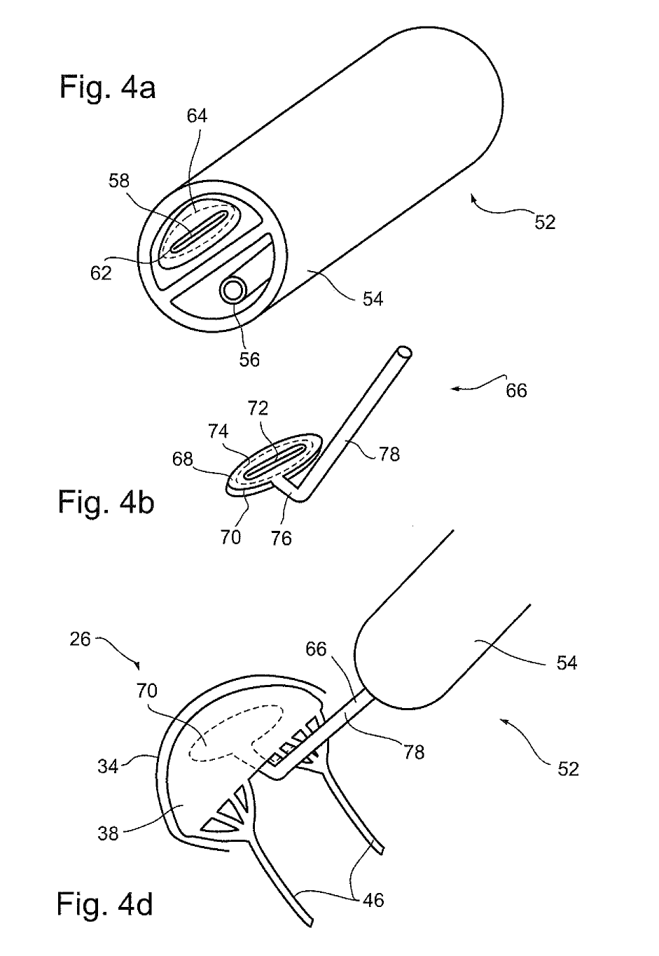

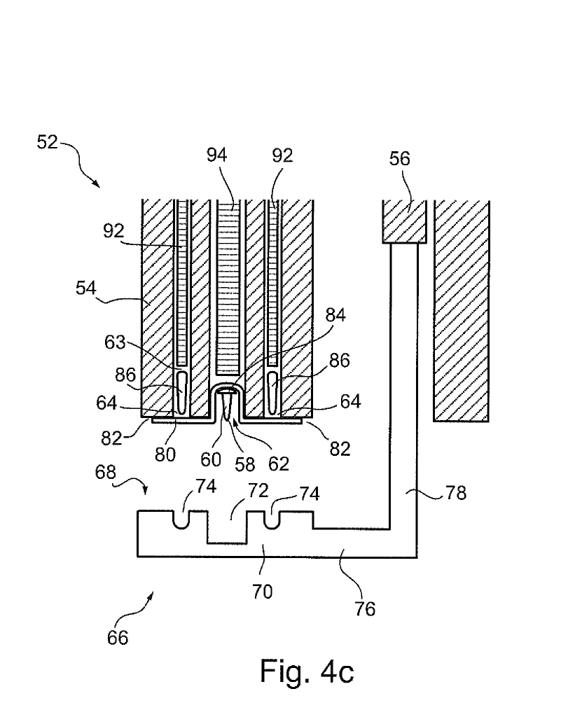

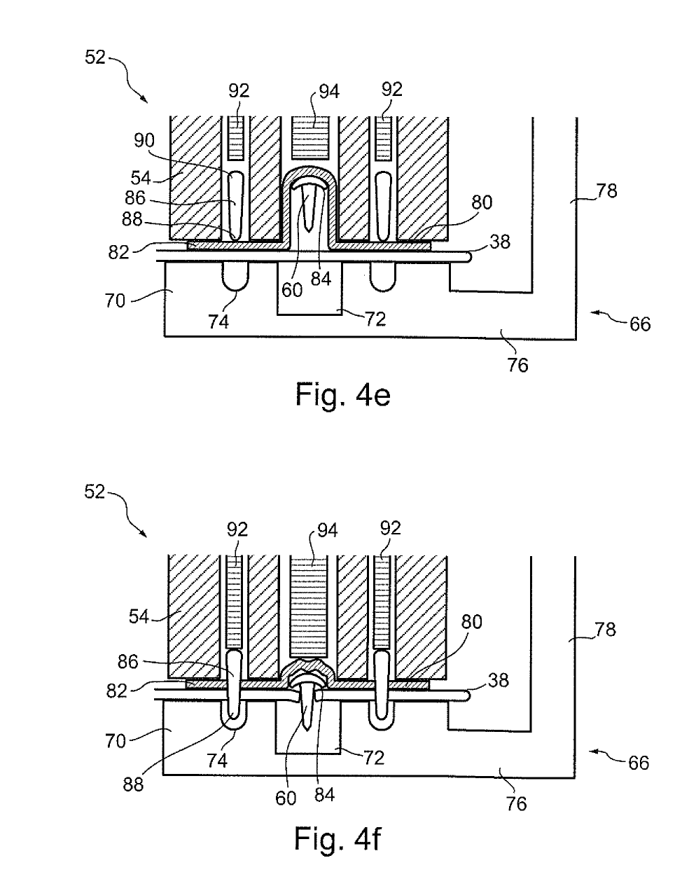

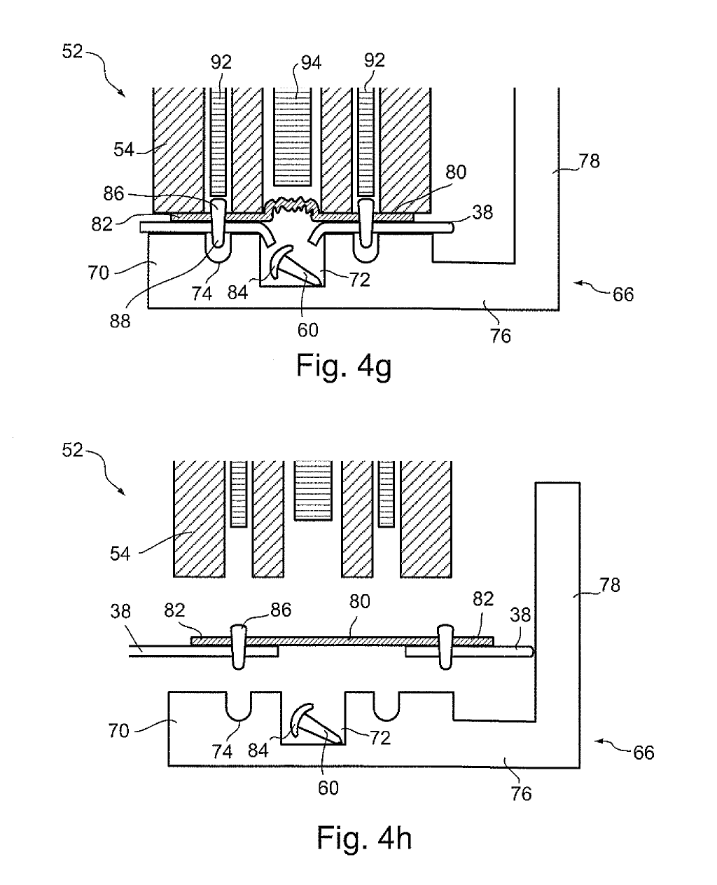

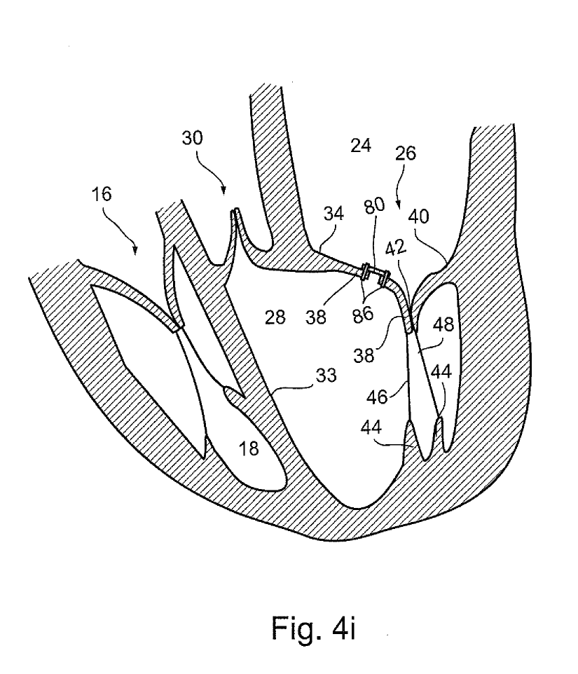

A first embodiment of the present invention relates to augmentation of membrane such as a cardiac leaflet, for example a posterior mitral valve leaflet. An embodiment of membrane augmentation in accordance with a method of the present invention is discussed with reference to FIGS. 4A-4I.

An embodiment of a surgical fastening device, such as a membrane augmenting stapler 52 depicted in FIGS. 4A-4I, is provided allowing quick cardiac leaflet augmentation where, with one simple stapling action a leaflet is slit and a periphery of an implant that acts as a patch secured to the periphery of the slit.

In FIG. 4A is depicted a distal end of stapler body 54 of membrane augmenting stapler 52 from below showing an anvil attachment tube 56, a cutting edge 58 of a cutting blade 60 positioned inside an implant container 62 and an oblate elliptical staple ejection ring 64 surrounding implant container 62.

In FIG. 4B is depicted an anvil 66 of membrane augmenting stapler 52 viewed from above. On a top surface 68 of an anvil head 70 is apparent blade-accepting slot 72 surrounded by clinching groove ring 74. Attached to anvil head 70 by crosspiece 76 is anvil stem 78 configured to reversibly lockingly engage anvil attachment tube 56 of stapler body 54.

In FIG. 4C is depicted anvil 66 coupled to stapler body 54 in cross-section where stem 78 engages anvil attachment tube 56 so that blade-accepting slot 72 is positioned across from cutting blade 60 and clinching groove ring 74 is positioned across staple ejection ring 64. Held inside implant container 62 and surrounding cutting blade 60 is patch 80 of tissue as a membrane augmenting implant, which edges 82 project outwards past staple ejection ring 64 to be held flush against the bottom face of distal end 54.

Distal end of stapler body 54 is circular and configured to fit inside a mitral valve lumen, having a diameter of about 3 cm.

Cutting blade 60 is a 3 mm high, 2 cm long and 0.3 mm thick stainless steel strip sharpened to define a 50 micron broad cutting edge 58. Fixed (e.g., by laser welding) on the side of cutting blade 60 opposing cutting edge 58 is curved implant shield 84 fashioned of a 0.5 mm thick, 2 cm long and 2 mm broad sheet of stainless steel.

Inside stapler magazine 63 which opens out to constitute staple ejection ring 64 are held plurality (e.g., 12) staples 86 of 0.5 mm broad 0.5 mm thick stainless steel wire bent in a square U-shape with legs pointing towards staple ejection ring 64 and with crowns of staples 86 contacting a drive blade 92 of membrane augmenting stapler 52.

Patch 80 is a 1 cm wide by 2.5 cm long patch of commercially-available cross-linked bovine pericardium pushed into implant container 62 between a knife driver 94 and implant shield 84 of cutting blade 60 so that cutting blade 60 is contained within implant container 62 and nestled in patch 80. Patch 80 is held flush with the distal end of stapler body 54 with the help of a tacky biocompatible adhesive (as well known in the art, see for example U.S. Pat. No. 6,126,919 or 6,325,810). It is seen that patch 80 is buckled to accommodate cutting blade 60.

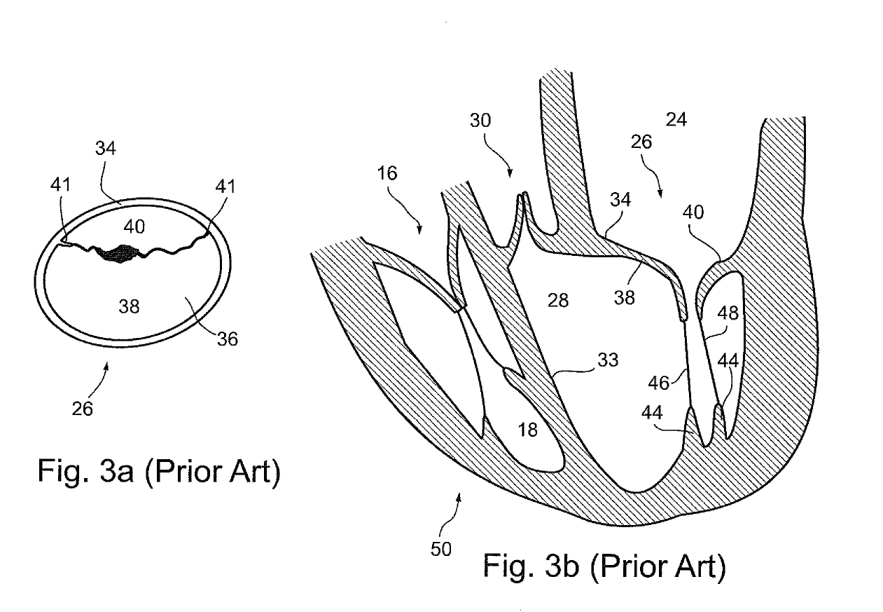

For use, a mitral valve 26 of a subject having ischemic mitral regurgitation, e.g., as depicted in FIG. 3, is exposed. For example, as described in Kincaid et al, mitral valve 26 is exposed through a median stemotomy, aortic and bicaval cannulation, moderate hypothermic cardiopulmonary bypass, and cold-blood retrograde cardioplegia. Alternative approaches include right or left thoracotomies. During the procedure, the heart may be fibrillating or arrested. Mitral valve 26 is exposed in the usual way, for example right-sided or left-sided left atriotomy, a transseptal incision with or without left atrial roof opening.

As depicted in FIG. 4D, anvil is maneuvered through mitral valve annulus 34 and between chordae 46 to be placed underneath anterior leaflet 38. Stem 78 engages anvil attachment tube 56 and is locked in place so that anterior leaflet 38 is located between stapler body 54 and anvil head 70.

In FIG. 4E, membrane augmenting stapler 52 is triggered in the usual way, pulling anvil head 70 and the distal end of stapler body 54 together to clamp anterior leaflet 38 therebetween. In such a way, patch 80 is placed against leaflet 38 so as to define an overlap region of patch 80 with leaflet 38. Cutting edge 58 of cutting blade 60 is directed towards the region circumscribed by the overlap region while legs 88 of staples 86 are directed towards the overlap region. Substantially simultaneously, drive blade 92 presses against crowns 90 of staples 86, forcing staples 86 out of the stapler magazine so that legs 88 to penetrate patch 80 near patch edges 82 in the overlap region and subsequently to pass through anterior leaflet 38. Substantially simultaneously, knife driver 94 presses against implant shield 84 through patch 80. Implant shield 84 prevents cutting blade 60 from damaging patch 80 and rather cutting blade 60 is forced downwards to slice into anterior leaflet 38.

In FIG. 4F, legs 88 of staples 86 are depicted having penetrated through patch 80 and anterior leaflet 38 to enter clinching groove ring 74 while cutting blade 60 is depicted having cut clear through anterior leaflet 38.

In FIG. 4G, as drive blade 92 and knife driver 94 descend further, cutting blade 60 is pushed down to fall into blade accepting slot 72 while legs 88 of staples 86 are bent by contact with clinching groove ring 74 to curve inwards, forcing staples 86 into a loop shape that substantially clamps and holds together anterior leaflet 38 and patch 80.

In FIG. 4H, the trigger of membrane augmenting stapler 52 is released, allowing stapler body 54 and anvil head 70 to move apart and away from anterior leaflet 38 augmented with patch 80. Anvil 66 is disengaged from stapler body 54 and maneuvered out from between chordae 46 and out through mitral lumen 36 together with cutting blade 60.

As depicted in FIG. 4I, the augmentation of anterior leaflet 38 improves coaptation, in analogy to the disclosed in Kincaid et al.

In embodiments, a mitral valve annulus supporting device such as an annuloplasty ring is deployed to reshape the mitral annulus in addition to augmentation of the valve leaflet as discussed above, whether prior or subsequent to the augmentation.

An embodiment of the teachings of the present invention has been discussed above with reference to augmentation of an anterior leaflet 38 of a mitral valve 26 as such embodiments may prove useful in treating ischemic mitral regurgitation, a common pathological condition. In non-depicted embodiments other cardiac leaflets are augmented in an analogous fashion, such as a mitral valve posterior leaflet 40 or leaflets of other cardiac valves such as of a tricuspid valve 16, a semi-lunar valve 20 or an aortic semi-lunar valve 30. In embodiments a membrane other than a cardiac valve is augmented.

Membrane augmenting stapler 52 discussed above is configured to produce and patch a single straight slit in a membrane, for example by the fact that cutting blade 60 is straight. In non-depicted embodiments, a membrane augmenting fastening device of the present invention is configured to produce and patch a curved slit, for example by including a curved cutting blade. A mitral valve leaflet that is augmented in accordance with the teachings of the present invention with the help of an implant secured to patch a curved slit is forced to buckle upwards which, in embodiments, provides an exceptionally high degree of coaptation.

Membrane augmenting stapler 52 discussed above is configured to augment a membrane such as a posterior leaflet of a mitral valve with an asymmetrically oblate elliptical graft, for example, by the fact that staple ejection ring 64 defines an asymmetrically oblate elliptical closed curve. In non-depicted embodiments, a membrane augmenting fastening device of the present invention is configured to augment a membrane with an implant having an alternative shape. Alternative shapes include, but are not limited to, circles, ellipses, ovals, ovoids as well as symmetrically and asymmetrically oblate ovals, ellipses or circles.

In some cases, it is not convenient for a medical professional such as a surgeon to associate, immediately before a surgical procedure, an implant such as 80 and a cutting blade such as 60 with a fastening device 52 as described above. In embodiments, a cartridge comprising a cartridge body, a staple magazine including a staple ejection ring loaded with staples, and an implant container containing an implant and a cutting blade, substantially as described above is provided, that is to say a cartridge comprising an implant container containing an implant surrounding a cutting blade so that a cutting edge of the blade faces outwards from the container In embodiments, such a cartridge also comprises a knife driver and/or a staple drive blade. Preferably, such a cartridge is provided packaged and sterile for use. Such a cartridge is configured to be secured to a properly configured fastening device and then used substantially as described above.

### Stapler Useful for Cardiac Valve Attachment

As noted above and depicted in FIG. 3, in a heart 50 suffering from ischemic mitral regurgitation mitral valve 26 and associated chordae 46 and 48 are patent. The insufficient coaptation of leaflets 38 and 40 that leads to the regurgitation of blood is a result of deformation of mitral valve annulus 34 and misdirected pulling forces applied through chordae 46 and 48 to leaflets 38 and 40, both resulting from necrosis and consequent deformation of the wall of left ventricle 28. In such cases, the regurgitation may be treated by improving leaflet coaptation.

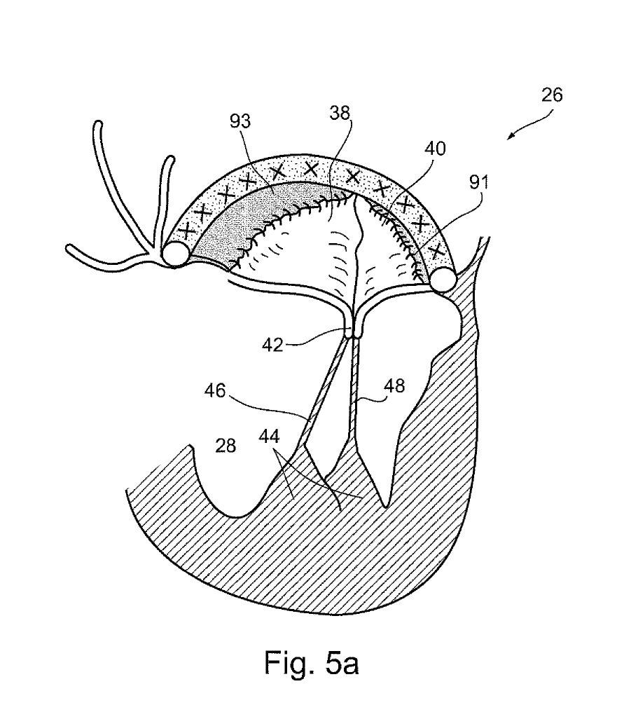

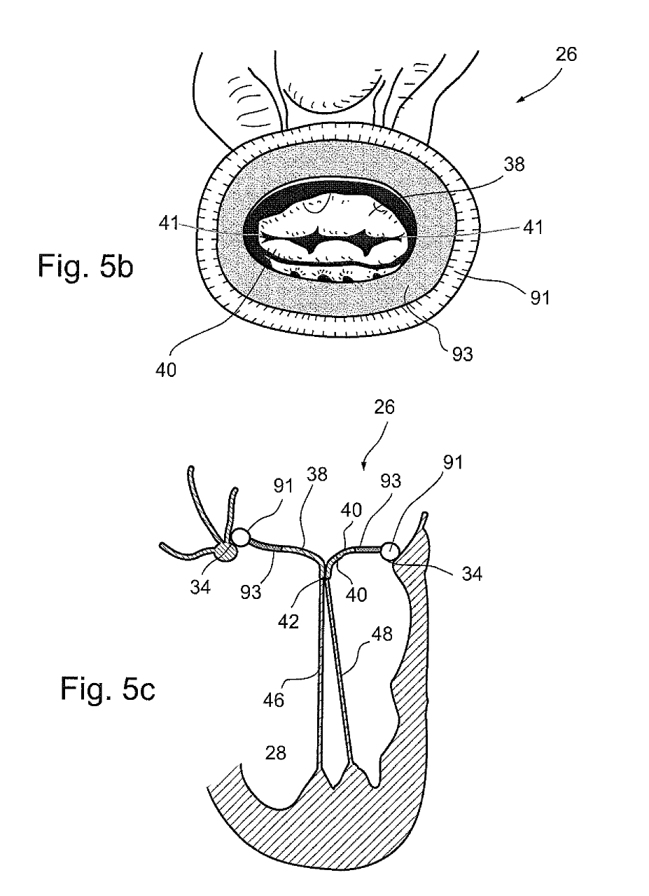

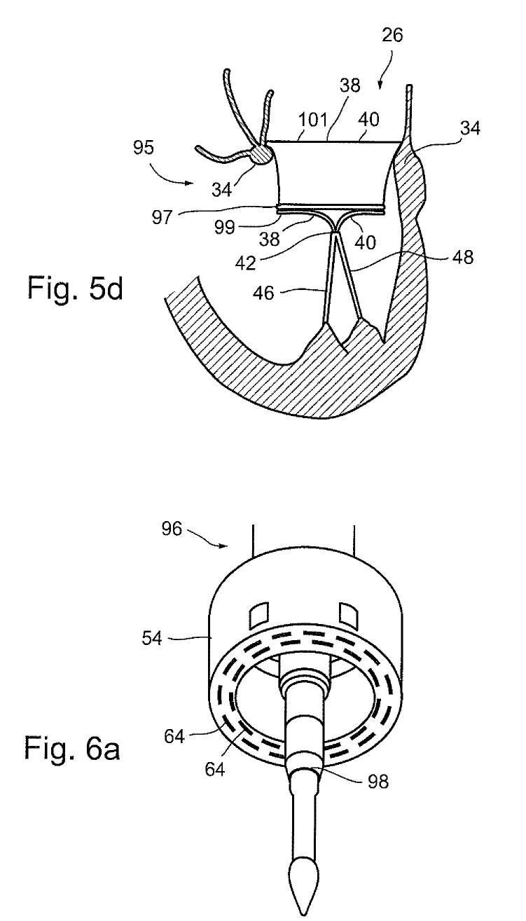

In copending PCT patent application identified by Attorney Docket No. 39351 of the Inventor are disclosed annuloplasty rings including a membrane that at least partially covers the lumen of the annuloplasty ring. Therein is taught, and as is depicted in FIG. 5A, that a mitral valve leaflet 38 is detached, an annuloplasty ring 91 with an attached membrane 93 implanted in the usual way, attached membrane 93 trimmed to cover a sector defined by a chord having a length approximately that of detached leaflet 38, and leaflet 38 reattached to membrane 93 effectively augmenting leaflet 38, that in embodiments such as depicted in FIG. 5A improves leaflet coaptation 42.

In copending PCT patent application identified by Attorney Docket No. 39351 of the Inventor are also disclosed annuloplasty rings including an annular membrane that at least partially covers the lumen of the ring and has a central void through the membrane defining a lumen. Therein is taught and as is depicted in FIG. 5B, that a mitral valve 26 is detached intact from a respective mitral valve annulus, so as to leave the valve leaflets 38 and 40 associated through commissures 41. An annuloplasty ring 91 is implanted in the usual way. Membrane 93 is trimmed so that the lumen therethrough is approximately the size of detached mitral valve 26. The periphery of mitral valve 26 is secured near an inner rim of membrane 93. In such a way, the tissue surrounding mitral valve 26 is augmented and mitral valve 26 displaced and allowed to settle downwards into left ventricle 28, which allows realignment of leaflets 38 and 40 that, in embodiments such as depicted in FIG. 5C, improves leaflet coaptation 42.

In copending PCT patent application identified by Attorney Docket No. 39351 of the Inventor a similar augmentation of tissue surrounding a mitral valve is achieved with a substantially tubular implant which includes an annuloplasty ring at neither, either or both a proximal end (attached to mitral valve annulus) or distal end (to which mitral valve is attached). In FIG. 5D is depicted tubular implant 95 including an annuloplasty ring 97 at distal end 99 to which mitral valve 26 is attached and not at proximal end 101 attached to mitral valve annulus 34.

The three above-discussed procedures, as well as other procedures known in the art such as the implantation of prior art annuloplasty rings, require placing many sutures around the entire periphery of both a mitral valve annulus and of the outer edge of the circular implant. Further, for the procedures taught in copending PCT patent application identified by Attorney Docket No. 39351 where an intact mitral valve is displaced, implantation requires placing many sutures around the periphery of the mitral valve and the inner rim of the membrane or the distal end of the tubular implant. Such suturing is difficult and time consuming. It is not simple to tension all sutures equally. It is not simple to evenly distribute the sutures about the mitral valve, the mitral valve annulus and/or the implant such that there is no deformation. Thus, it is possible that attachment will be incorrect providing less than ideal results which may lead, for example, to leakage.

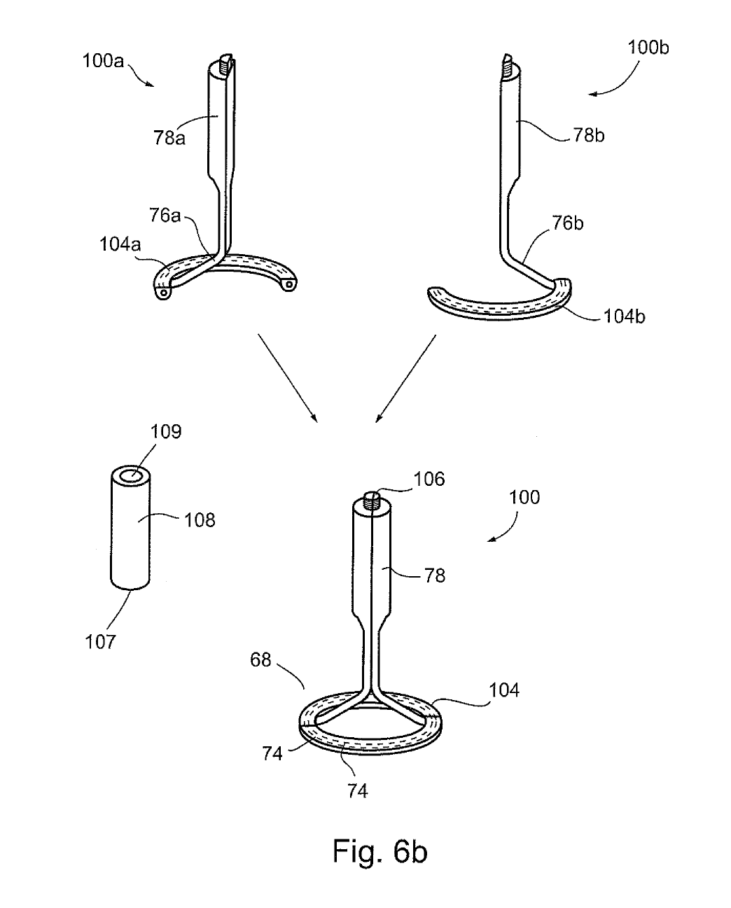

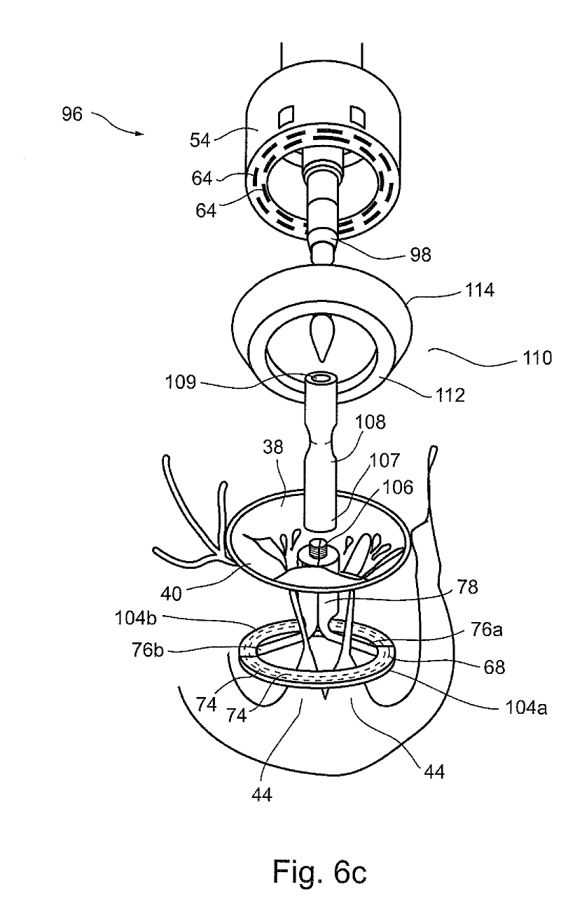

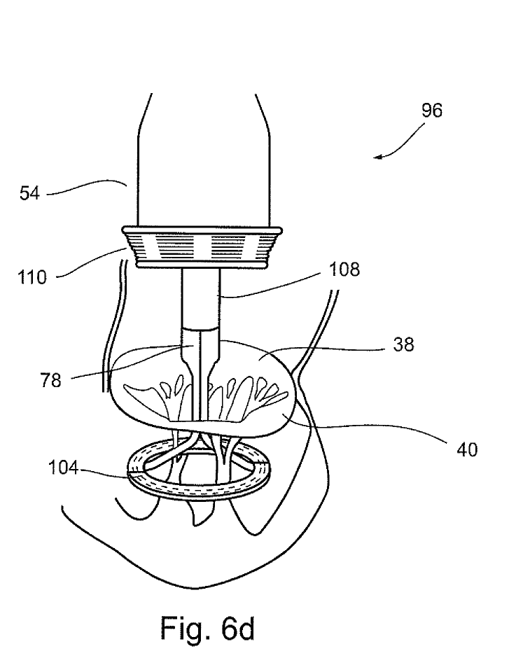

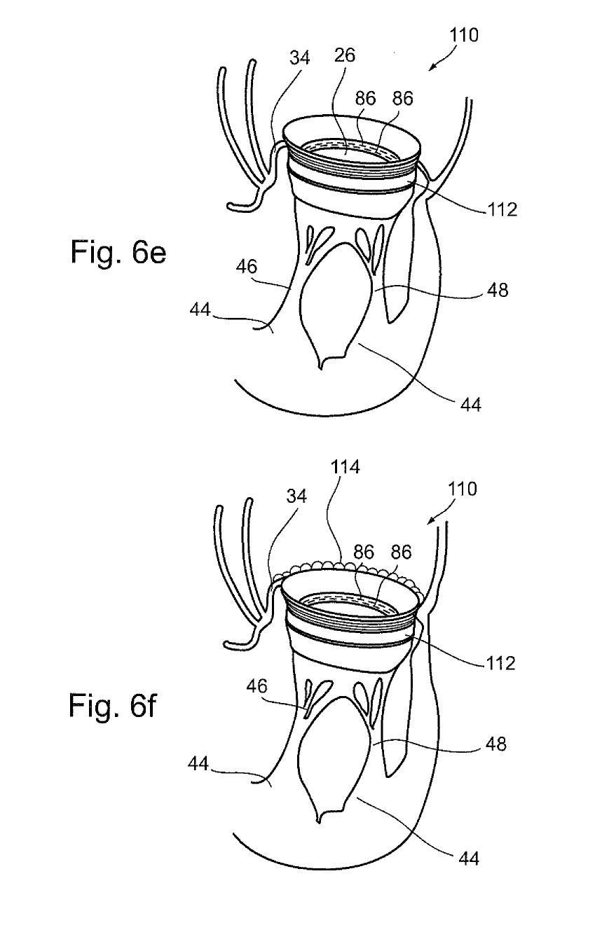

In an embodiment of the present invention, a mitral valve periphery stapler is provided allowing quick and/or simple and/or accurate securing of a mitral valve (e.g., an intact mitral valve) to an implant (such as an annular graft, an annuloplasty ring as described above or a tubular implant as described above or the like). The utility of a mitral valve periphery stapler of the present invention results, in part, from an anvil head which allows maneuvering of the anvil head between the mitral valve chordae. An embodiment of such a mitral valve periphery stapler useful for securing a mitral valve detached from a respective mitral valve annulus to an implant such as taught in copending PCT patent application identified by Attorney Docket No. 39351 is discussed with reference to FIGS. 6A-6F.

In FIG. 6A is depicted a distal end of stapler body 54 of mitral valve periphery stapler 96 from below showing two concentric circular staple ejection rings 64 surrounding an anvil-engaging pin 98.

In FIG. 6B are depicted the two parts 100a and 100b of a two-part anvil 100 of mitral valve periphery stapler 96, separated (above) as well as assembled to constitute a single anvil (below). First part 100a of anvil 100 of mitral valve periphery stapler 96 includes a portion 78a of stem 78, a crosspiece 76a protruding at a 300 angle downwards from perpendicular from stem portion 78a to attach to one end of anvil head portion 104a. Second part 100b of anvil 100 of mitral valve periphery stapler 96 includes a portion 78b of stem 78, a crosspiece 76b protruding at a 300 angle downwards from perpendicular from stem portion 78b to attach to one end of anvil head portion 104b. Each of anvil head parts 104a and 104b constitute a 180° sector of anvil head 104.

Anvil parts 100a and 100b are configured to mate, together constituting an anvil 100 having a circular planar anvil head 104. When mated, on top surface 68 of anvil head 104 are apparent two concentric clinching groove rings 74. When mated, threads 106 are constituted at the end of stem 78. A distal end 107 of a stem extension piece 108 is configured to engage threads 106 holding anvil parts 100a and 100b together. Further, a proximal end 109 of stem extension piece 108 is configured to reversibly lockingly engage anvil engaging pin 98.

Analogously to the discussed above for membrane augmenting stapler 52, when anvil 100 (assembled by joining anvil parts 100a and 100b) and stapler body 54 of mitral valve periphery stapler 96 are associated through stem extension piece 108 by engaging anvil engaging pin 98 with proximal end 109 of stem extension piece 108 and by screwing distal end 107 of a stem extension piece 108 over threads 106 of anvil 100, clinching groove rings 74 are positioned across staple ejection rings 64.

In embodiments, staple ejection rings 64 are circular and of a diameter that is suitable for fastening a desired implant to the periphery of an intact detached mitral valve 26 as is discussed below (e.g., 45 mm). Consequently, the distal end of stapler body 54 is generally small enough to pass through a mitral valve annulus.

Analogously to the discussed above with reference to membrane augmenting stapler 52, inside both staple magazines 63 opening out to constitute staple ejection rings 64 of mitral valve periphery stapler 96 are held a plurality of staples 86 of 1 mm broad 0.5 mm thick stainless steel wire bent in a square U-shape with legs 88 pointing towards a staple ejection ring of a staple ejection ring 64 and with crowns 90 of staples 86 contacting one of two drive blades 92 of mitral valve periphery stapler 96, each drive blade associated with one of the two stapler magazines 63 and staple ejection rings 64.

For use, a mitral valve 26 of a subject having ischemic mitral insufficiency, e.g. as depicted in FIG. 3, is exposed in the usual way as discussed above.

Mitral valve 26 is detached intact from mitral valve annulus 34, so as to leave the valve leaflets 38 and 40 associated through commissures 41 with subvalvular apparatus including chordae 46 and 48 intact. Each of anvil head parts 104a and 104b of anvil 100 is separately passed through mitral valve annulus 34 and mitral valve lumen 36 and maneuvered with a rotating motion around chordae 46 and 48 so that stem portions 78a and 78b remain in left atrium 24.

In FIG. 6C, anvil parts 100a and 100b are mated so as to constitute an assembled anvil 100 where chordae 46 and 48 are encircled by annular anvil head 104. Stem extension piece 108 is screwed onto stem 78 to engage threads 108 so as to hold anvil 100 together. A tubular implant 110 such as taught in copending PCT patent application identified by Attorney Docket No. 39351 and similar to implant 95 depicted in FIG. 5D is placed over stem extension piece 108 and stem 78 of anvil 100 so that a distal end 112 is nearer anvil head 104 than a proximal end 114.

In FIG. 6D, anvil engaging pin 98 of stapler body 54 is coupled to the distal end of stem extension piece 108 so that the periphery of mitral valve 26 and distal end 112 of tubular implant 110 is located between stapler body 54 and anvil head 100.

Once assembled as depicted in FIG. 6D, mitral valve periphery stapler 96 is triggered in the usual way, pulling anvil head 100 and the distal end of stapler body 54 together to clamp the periphery of mitral valve 26 therebetween. Substantially simultaneously, the two drive blades 92 press against crowns 90 of staples 86, forcing legs 88 to penetrate tubular implant 110 and to pass through mitral valve 26.

Analogously to the discussed above with reference to membrane augmenting stapler 52, legs 88 of staples 86 pass through tubular implant 110 and mitral valve 26. and are bent inwards by contact with clinching groove rings 74 to curve inwards, forcing staples 86 into a loop shape that substantially clamps and holds together tubular implant 110 and mitral valve 26. Mitral valve periphery stapler 96 is released, allowing stapler body 54 and anvil head 100 to move apart. Stem extension piece 108 is disengaged from stapler body 54 and unscrewed from stem 78. Anvil head 104 is taken apart and anvil head parts 104a and 104b removed through lumen 36 of mitral valve 26.

In FIG. 6E, mitral valve 26 is depicted secured to distal end 112 of tubular implant 110 with two concentric circles of staples 86.

In FIG. 6F, proximal end 114 of tubular implant 110 is depicted secured to mitral valve annulus 34 in the usual way with sutures. As depicted above in FIG. 5C, such displacement of mitral valve 26 improves mitral valve leaflet coaptation.

As discussed above, mitral valve 26 is first detached from mitral valve annulus 34 and subsequently secured to an implant (distal end 112 of tubular implant 110). In embodiments, a mitral valve is first secured to an implant, for example in accordance with the teachings of the present invention, and subsequently detached from a mitral valve annulus.

As discussed above, distal end 112 of tubular implant 110 is first secured to a mitral valve 26 and subsequently proximal end 114 is secured to mitral valve annulus 34. In embodiments, a proximal end of a tubular implant is first secured to a mitral valve annulus and subsequently a distal end of a tubular implant is secured to a mitral valve.

Mitral valve periphery stapler 96 discussed above is configured and used to attach the annular distal end 112 of tubular implant 110 such as disclosed in copending PCT patent application identified by Attorney Docket No. 39351 to the periphery of an intact mitral valve detached from a respective mitral valve annulus. In non-depicted embodiments, a mitral valve periphery stapler such as 96 is used to attach the inner rim of an annular implant such as disclosed in copending PCT patent application identified by Attorney Docket No. 39351 or the inner rim of an annular graft (e.g., a ring of tissue) to the periphery of an intact mitral valve detached from a respective mitral valve annulus, substantially in accordance with the teachings of copending PCT patent application identified by Attorney Docket No. 39351. In analogy to the discussed immediately hereinabove, in embodiments, an outer periphery of an annular implant is first secured to a mitral valve annulus and subsequently an inner rim is secured to a mitral valve.

In mitral valve periphery stapler 96 discussed above, annular anvil head 104 of anvil 100 is split into two parts 104a and 104b, each defining an equal 180° sector of annular anvil head 104. In embodiments, each part of an annular anvil head defines a different sized sector.

In mitral valve periphery stapler 96 discussed above, annular anvil head 104 of anvil 100 is split into two parts 104a and 104b. In embodiments, an annular anvil head is split into more than two parts, e.g., three, four, five, six or even more parts.

In mitral valve periphery stapler 96 discussed above, stem 78 of anvil 100 is split and is made up of two parts 78a and 78b that are parts of anvil parts 100a and 100b, respectively. In embodiments, a stem of a multi-part anvil is a single piece that is not split, see below.

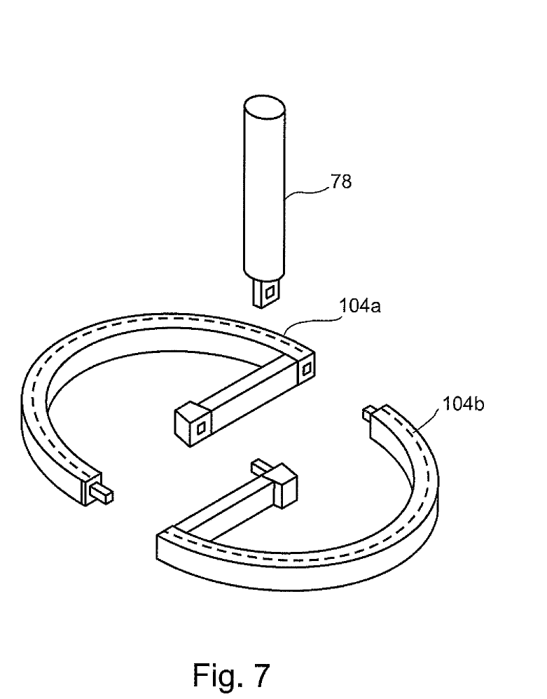

In mitral valve periphery stapler 96 discussed above, parts of stem 78 of anvil 100 are a part 100a or 100b of anvil 100 that comprises a part 104a or 104b of anvil head 104. In embodiments, a stem is a separate component that is not a part of an anvil that comprises a part of an anvil head, as depicted in FIG. 7.

In mitral valve periphery stapler 96 discussed above, crosspieces 76a or 76b protrude at a 30° angle downwards from perpendicular from a respective stem portion 78a or 78b to attach to one end of anvil head portion 104a or 104b. In embodiments, crosspieces protrude downwards from perpendicular at an angle different from 30°. In embodiments, crosspieces protrude upwards from perpendicular. In embodiments, crosspieces protrude substantially perpendicularly from stem 78, see below.

In mitral valve periphery stapler 96 discussed above, top surface 68 of anvil head 104 defines a plane substantially perpendicular to stem 78. In embodiments, a plane defined by a top surface of an anvil head is not perpendicular to a respective stem but is tilted, in embodiments up to about 30° and even up to about 60° relative to the stem. In embodiments, such tilting makes maneuvering of the anvil head parts through a mitral valve lumen more convenient.

In mitral valve periphery stapler 96 discussed above, top surface 68 of anvil head 104 is substantially planar. In embodiments, a top surface of an anvil head is not planar. For example, in embodiments, top surface 68 has a saddle contour similar to that of a patent mitral valve annulus.

In mitral valve periphery stapler 96 discussed above, crosspieces 76a and 76b attach to one end of an anvil head portion 104a and 104b respectively. In embodiments, a crosspiece attaches to a respective anvil head portion not at an end of an anvil head portion rather, for example, in the middle of a respective anvil head portion, see below.

In mitral valve periphery stapler 96 discussed above, anvil head 104 and stapler body 54 are configured to attach an implant with the help of two concentric circular rows of staples. In embodiments, a stapler of the present invention is configured to attach an implant with the help of one row of staples, as depicted in FIG. 7. In embodiments, a stapler of the present invention is configured to attach an implant with the help of three or more rows of staples.

In mitral valve periphery stapler 96 discussed above, anvil head 104 and stapler body 54 are configured to attach an implant with the help of substantially circular rows of staples. In embodiments, an anvil head and stapler body are configured to attach an implant with the help of one or more rows of staples that define a closed curve that is not a circle, for example an oval, an ellipse, an ovoid, or a symmetrically or asymmetrically oblate circle, oval or ellipse.

### Stapler Useful for Augmentation of Tissue Around a Cardiac Valve

As noted above and depicted in FIGS. 5B and 5C, in copending PCT patent application identified by Attorney Docket No. 39351 is disclosed the augmentation of the tissue around an intact mitral valve allowing displacement of the mitral valve with the help of a substantially annular membrane. As noted above and depicted in FIGS. 5D and 6F, in copending PCT patent application identified by Attorney Docket No. 39351 is disclosed the augmentation of the tissue around an intact mitral valve allowing displacement of the mitral valve with the help of a substantially tubular implant. In both cases, the displacement allows the mitral valve to settle downwards into the left ventricle, which allows realignment of the leaflets and chordae that in embodiments improves leaflet coaptation as depicted in FIGS. 5C and 5D

In embodiments of the present invention, features discussed hereinabove are combined in a single fastening device to allow simple and/or repeatable augmentation of the tissue surrounding a cardiac valve as discussed above. Specifically, a fastening device is provided including a cutting blade configured to produce a cut in the shape of a closed curve that is contained between two rows of lockable fasteners such as staples, each row defining a closed curve. With one action, e.g., one triggering of the fastening device:

1) native leaflets of a valve are attached to one edge of an implant (e.g., an inner edge of an annular implant or a distal end of a tubular implant);

2) another edge of an implant (e.g., an outer edge of an annular implant or a proximal end of a tubular implant) is attached to the native valve annulus or tissue near the annulus; and

3) the native leaflets are detached from the native annulus.

An embodiment of a surgical fastening device allowing augmentation of tissue surrounding a cardiac valve in accordance with the teachings of the present invention, stapler 116 is discussed with reference to FIGS. 5A-8J.

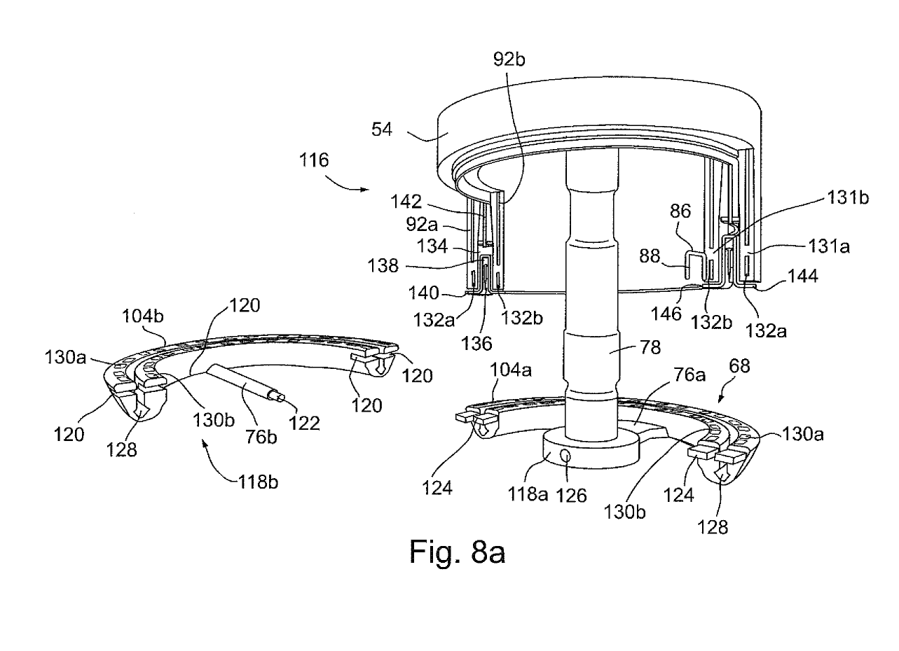

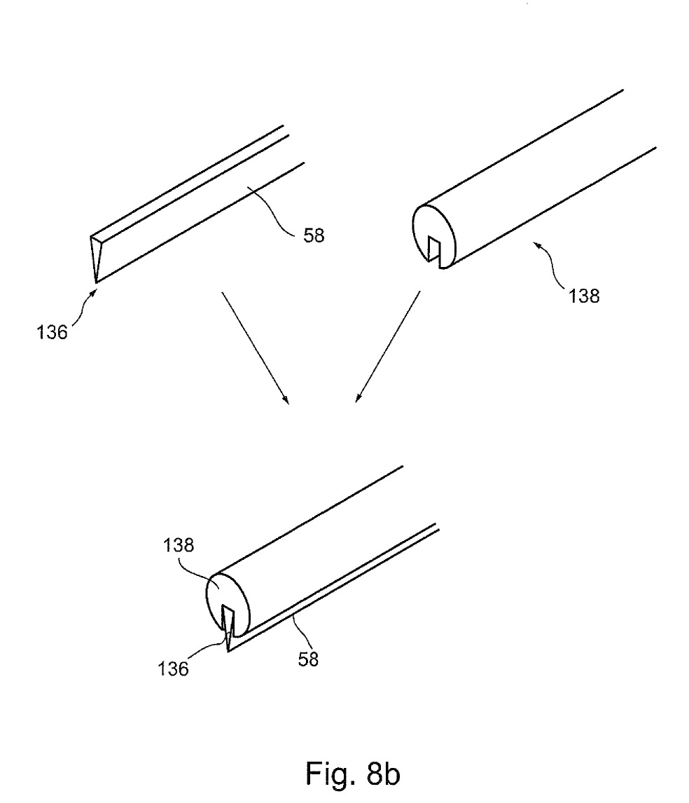

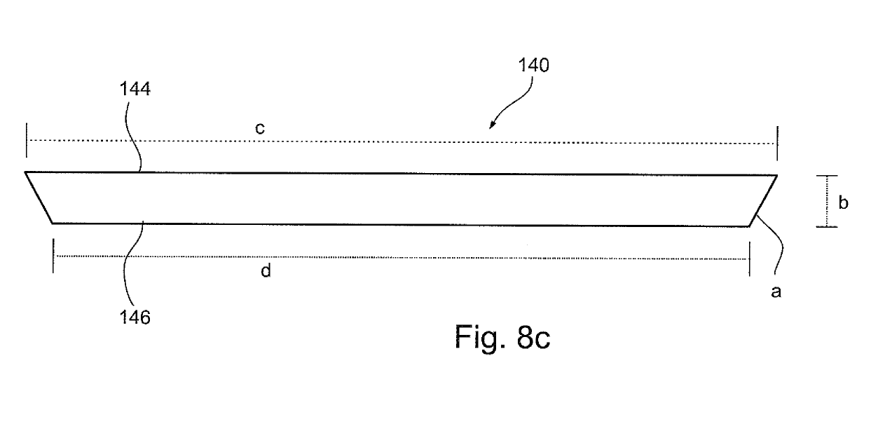

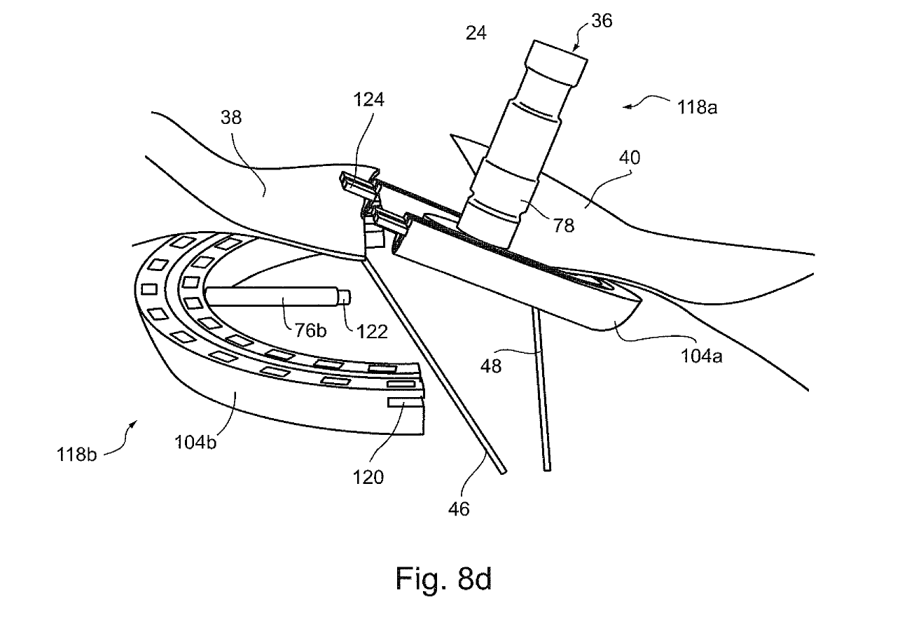

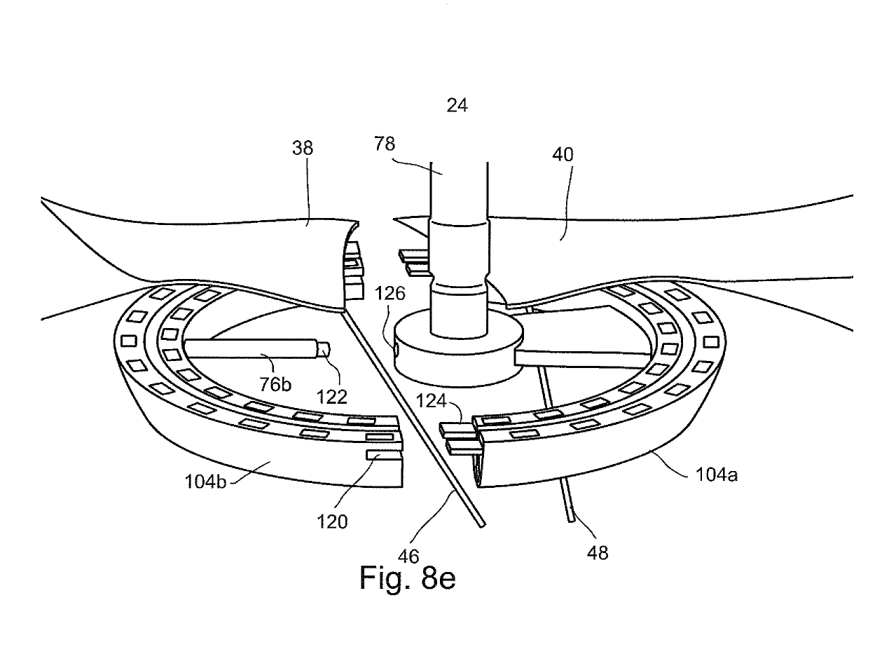

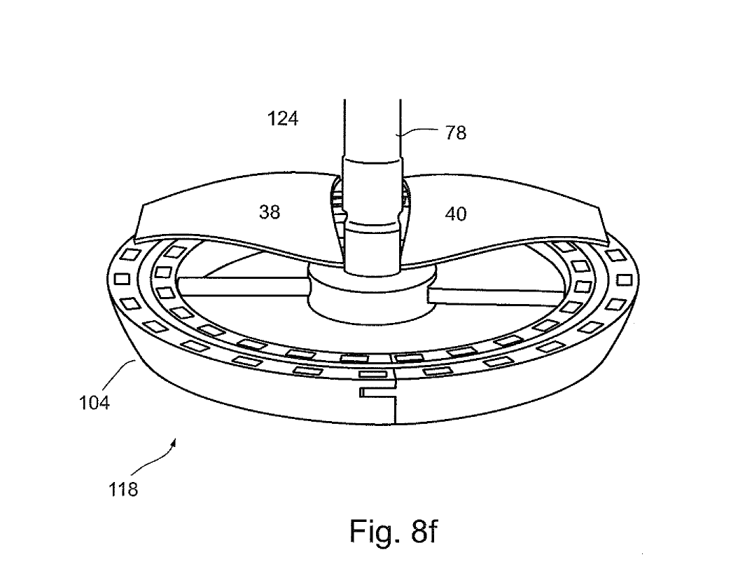

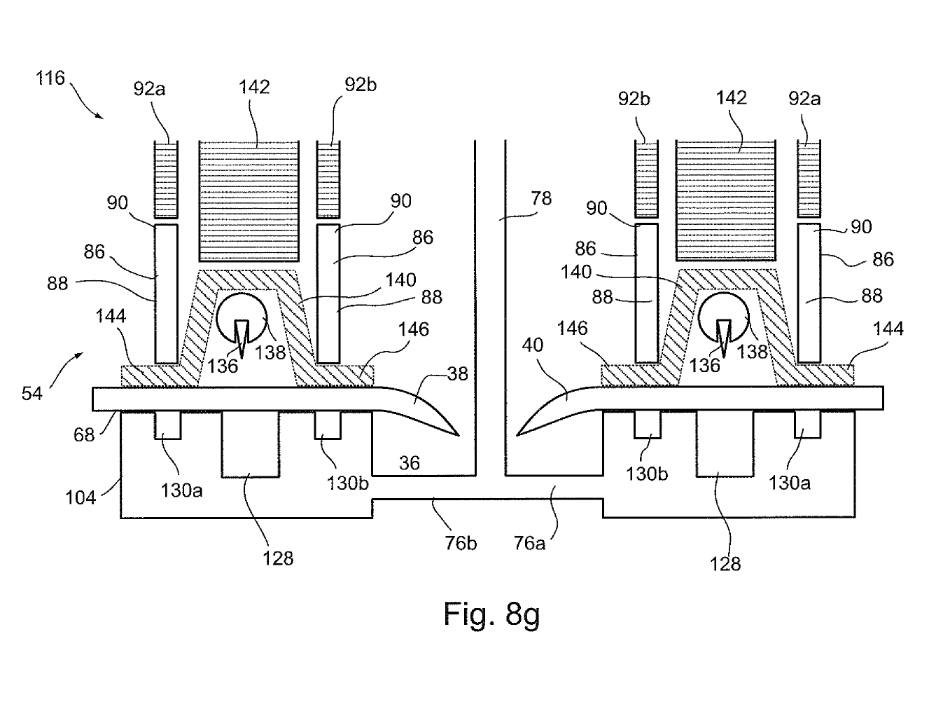

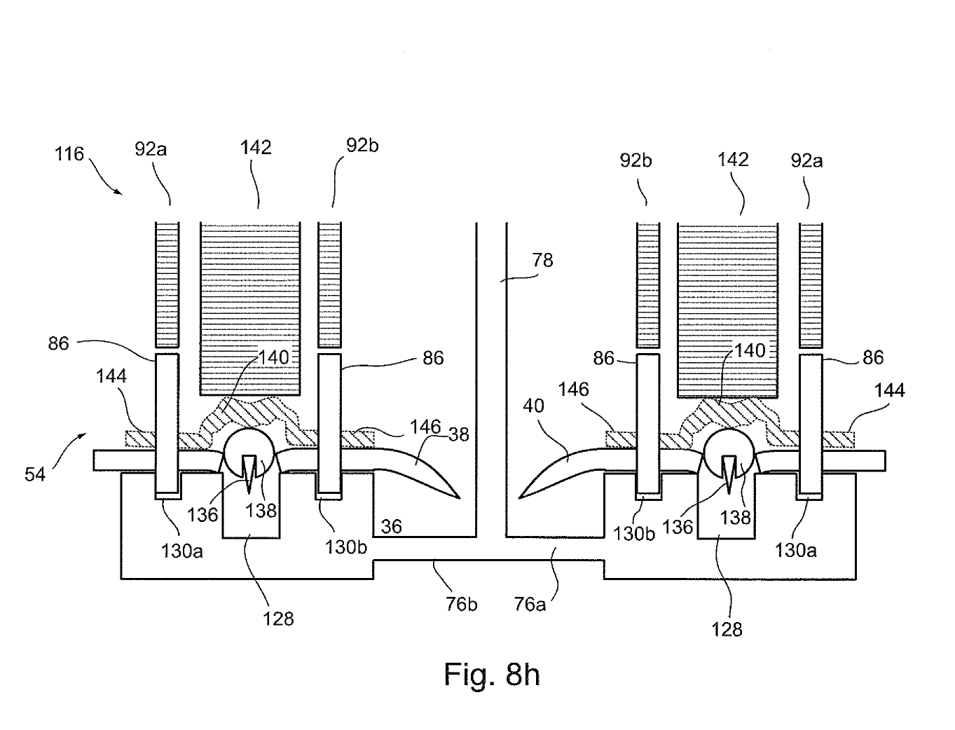

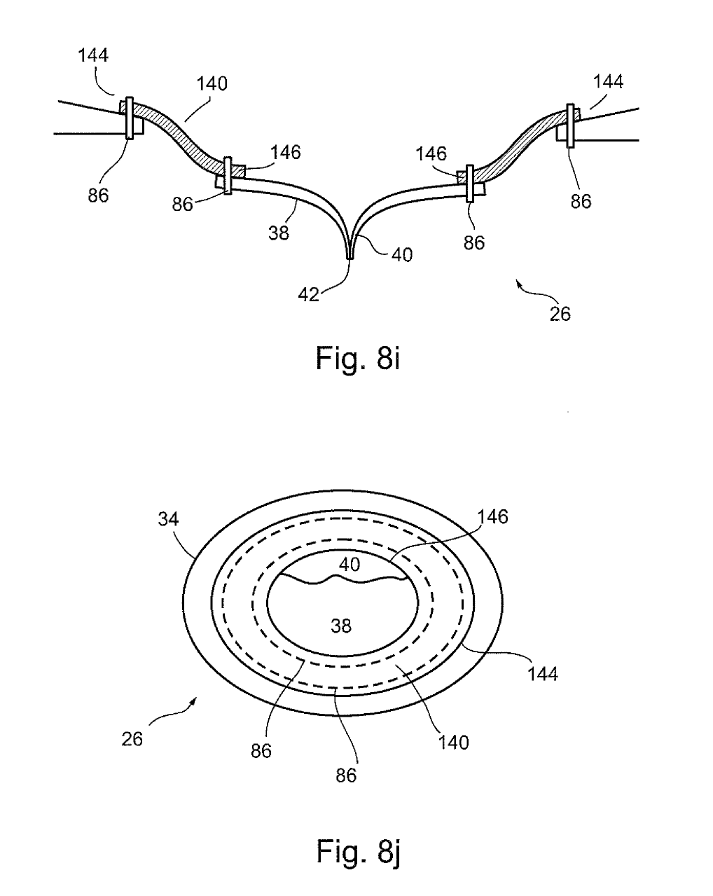

In FIG. 8A is depicted a distal end of stapler body 54 of stapler 116 in partial cross section engaged with a first part 118a of two-part anvil 118 and a second part 118b of anvil 118 not attached thereto.

First part 118a of anvil 118 includes a one-piece stem 78 from which protrudes a crosspiece 76a which connects to the middle of a first semicircular part 104a of anvil head 104 so that top surface 68 of anvil head 104 is perpendicular to stem 78. Second part 104b of anvil head 104 substantially comprises a crosspiece 76b which connects to the middle of a semicircular part 104b of anvil head 104. Second part 118b includes two slots 120 and a peg 122 which are configured to slidingly engage, respectively, tabs 124 and a hole 126 in first part 104a of anvil head 104. Both anvil-head parts 104a and 104b are semicircular and comprise a 180° sector of assembled anvil head 104. Apparent on top surface 68 of anvil head parts 104a and 104b is an outer staple clinching groove ring 130b, encircling a circular blade accepting slot 128, encircling an inner staple clinching groove ring 130b.

In stapler body 54 are seen an outer staple magazine 131a opening out to constitute an outer staple ejection ring 132a (45 mm diameter) opposite outer staple clinching groove ring 130b, encircling circular implant container 134 (42.5 mm diameter) opposite circular blade accepting slot 128, encircling an inner staple magazine 131b opening out to constitute an inner staple ejection ring 132b (41.5 mm diameter) opposite inner staple clinching groove ring 130b. Distal end of stapler body 54 is circular and configured to fit inside a mitral valve lumen, having a diameter of about 50 mm.

Inside staple magazines 131a and 131b associated with staple ejection rings 132a and 132b are held a plurality of staples 86 of 0.5 mm broad 0.5 mm thick square cross-section stainless steel wire bent in a square U-shape with legs 88 pointing towards the opening of staple ejection rings 132a and 132b and with crowns 90 of staples 86 contacting drive blades 92a and 92b, respectively, of stapler 116.

Inside circular implant container 132 are cutting blade 136, implant shield 138 and a frustoconical graft 140, which contacts knife driver 142.

Cutting blade 136, FIG. 8B, is a 2 mm high and 0.5 mm thick 272 mm long flexible stainless steel strip sharpened to define a 50 micron broad cutting edge 58 and bent into a circle to fit inside circular implant container 134 so that the two ends of cutting blade 136 abut. Implant shield 138 is a 250 mm long flexible Nitinol wire with a 1.5 mm diameter and a 0.5 mm axial slot configured to accept cutting blade 136.

Frustoconical implant 140, FIG. 8C, is a 6 mm (dimension a) broad strip of commercially available 0.5 mm thick crosslinked serous tissue such as bovine pericardium fashioned in the usual way (e.g., with the help of adhesive or sutures) to form the frustoconical shape of implant 140 5.4 mm high (dimension b), a proximal end 144 of 46 mm (dimension c) and a distal end 146 of 40.5 mm (dimension d).

As seen in FIG. 8A, frustoconical implant 140 is pushed into implant container 134 between a knife driver 142 on one side and implant shield 138/cutting blade 136 on another side so that implant 140 is buckled and cutting blade 136 nestled therein. Proximal end 144 of implant 140 projects outwards past outer staple ejection ring 132a. Distal end 146 of implant 140 projects inwards past inner staple ejection ring 132b. Ends 144 and 146 of implant 140 are held flush with the distal end of stapler body 54 with the help of a tacky biocompatible adhesive.

For use, a mitral valve 26 of a subject having ischemic mitral insufficiency, e.g., as depicted in FIG. 3, is exposed in the usual way as discussed above.

In FIGS. 8D and 8E, each of anvil parts 118a and 118b of anvil 118 is separately passed through mitral valve lumen 36 and maneuvered with a rotating motion around chordae 46 and 48 so that stem 78 remains in left atrium 24. Anvil parts 118a and 118b are mated by pushing tabs 124 into slots 120 and peg 122 into hole 126 so as to constitute an assembled anvil 118 where chordae 46 and 48 are encircled by annular anvil head 119, FIG. 8F.

Anvil 118 is engaged with stapler body 54 and locked in place so that anterior leaflet 38 and posterior leaflet 40 are located between stapler body 54 and anvil head 104.

In FIG. 5G, stapler 116 is triggered in the usual way, pulling anvil head 104 and the distal end of stapler body 54 together to clamp leaflets 38 and 40 therebetween.

In such a way, implant 140 is placed against mitral valve 26 so as to define two overlap regions of implant 140 with mitral valve 26, an outer overlap region closer to mitral valve annulus 34 with proximal end 144 of implant 140 and an inner overlap region closer to the center of mitral valve lumen 36 with distal end 146 of implant 140. The cutting edge of cutting blade 136 is directed towards the region between the two overlap regions. Legs 88 of staples 86 held in outer staple magazine 131a associated with outer staple ejection ring 132a are directed towards the outer overlap region. Legs 88 of staples 86 held in inner staple magazine 131b associated with inner staple ejection ring 132b are directed towards the inner overlap region.

In FIG. 8H, substantially simultaneously, drive blade 92a presses against crowns 90 of staples 86 held in outer staple magazine 131a associated with outer staple ejection ring 132a, forcing legs 88 to penetrate implant 140 near proximal end 144 and to pass through leaflets 38 and 40 in the outer overlap region. Substantially simultaneously, drive blade 92b presses against crowns 90 of staples 86 held in inner staple magazine 131b associated with inner staple ejection ring 132b, forcing legs 88 to penetrate implant 140 near distal end 146 and to pass through leaflets 38 and 40 in the inner overlap region. Substantially simultaneously, knife driver 142 presses against implant shield 138 through implant 140. Implant shield 138 prevents cutting blade 136 from damaging implant 140 and rather cutting blade 136 is forced downwards to slice into leaflets 38 and 40 between the two overlap regions.

Analogously to the discussed above for membrane augmentation stapler 52, legs 88 of staples 86 penetrate through leaflets 38 and 40 to enter clinching groove rings 130a and 130b while cutting blade 136 cuts clear through leaflets 38 and 40. Ultimately, cutting blade 136 and implant shield 136 fall into blade accepting slot 128 while legs 88 of staples 86 are bent by contact with clinching groove rings 130a and 130b to curve inwards, forcing staples 86 into a loop shape that substantially clamps and holds leaflet 38 and 40 together with implant 140.

Subsequently, the trigger of stapler 116 is released, allowing stapler body 54 and anvil head 104 to move apart and away from leaflets 38 and 40. Anvil 118 is disengaged from stapler body 54. Implant shield 138 and cutting blade 136 are dissociated and removed separately, for example with a forceps, from blade accepting slot 128 in anvil 118 and out through lumen 36 of mitral valve 26.

As depicted in FIG. 8I (side cross section), FIG. 8J (top view) and analogously to the depicted in FIGS. 5C and 5D, the tissue surrounding mitral valve 26 is augmented allowing mitral valve 26 to displace downwards into left ventricle 28, that improves coaptation 42 as discussed above with reference to mitral valve periphery stapler 96.

Stapler 116 discussed above is configured to augment the tissue surrounding a cardiac valve by excising the valve intact with a circular incision while substantially simultaneously securing an implant to the edges of the excision with two concentric rows of lockable fasteners, one encircling the incision and one encircled by the incision. In embodiments, (one or more of) the incisions and two rows of lockable fasteners are not concentric. In embodiments, (one or more of) the incisions and two rows of fasteners are not circular but have an alternatively-shaped closed curve. Alternative shapes include, but are not limited to, circles, ellipses, ovals, ovoids as wells as symmetrically and asymmetrically oblate ovals, ellipses or circles.

In embodiments of the method of the present invention, a mitral valve annulus supporting device such as an annuloplasty ring to reshape the mitral annulus is deployed to reshape the mitral annulus in addition to displacement of the valve, whether prior or subsequent to displacement of the valve in accordance with the teachings of the present invention.

In the embodiment discussed above, the teachings of the present invention have been discussed with reference to displacement of a mitral valve 26 as embodiments of the present invention may prove useful in treating ischemic mitral regurgitation, a common pathological condition. In non-depicted embodiments other cardiac valves such as of a tricuspid valve 16, a semi-lunar valve 20 or an aortic semi-lunar valve 30 may be displaced.

In the embodiment discussed above cutting blade 136 is a sharpened stainless steel strip sharpened to define cutting edge and flexible enough to bent into a circle to fit inside circular implant container 134. In embodiments, a cutting blade is a loop in the shape of a closed curve having sufficient flexibility to be collapsed to an elongated shape (e.g., long and narrow). In such embodiments, the cutting blade is removed from the left ventricle by collapsing to an elongated shape (for example, with forceps) and then pulling out through the mitral valve lumen.

In the embodiment discussed above, the teachings of the present invention have been discussed where a mitral valve is displaced by implantation of a frustoconical tubular implant. In non-depicted embodiments, other shaped implants are implanted including annular implants, tubular implants that are cylindrical and tubular implants that are not frustoconical (distal and proximal ends are not parallel). Generally a suitable implant includes a wall, the wall delimited by two edges each in the shape of a closed curve and defining a lumen.

In some cases, it is not convenient for a medical professional such as a surgeon to associate, immediately before a surgical procedure, an implant such as 140 and a cutting blade such as 136 with a fastening device such as 116 as described above. In embodiments, a cartridge comprising a cartridge body, an outer fastener magazine functionally associated with an outer fastener ejection ring holding a plurality of staplers directed towards the outer fastener ejection ring; surrounded by the implant container, an implant container in the shape of a closed curve at the distal end including an outwardly directed opening surrounded by the fastener ejection ring; surrounded by the implant container an inner fastener magazine functionally associated with an inner fastener ejection ring holding a plurality of staplers directed towards the inner fastener ejection ring where contained within the implant container, is an implant as described above where a first of the two edges extends outwards beyond the outer fastener ejection ring and a second of the two edges extends inwards beyond the inner fastener ejection ring; and a cutting blade contained within the implant container and nestled in the implant with a cutting edge directed towards the opening of the implant container. In embodiments, such a cartridge also comprises a knife driver and/or a staple drive blade associated with the inner staple magazine and/or a staple drive blade associated with the outer staple magazine. Preferably, such a cartridge is provided packaged and sterile for use. Such a cartridge is configured to be secured to a properly configured fastening device and then used substantially as described above.

It is important to note that a person weighing between 60 and 100 kg has a usual cardiac output of about 4 to 6 l blood/minute and about 15 blood/minute during maximum effort. It is known that a mitral valve lumen having a diameter of at least about 28 mm diameter is needed to transfer 15 l blood minute without undue stress. Thus, generally it is desirable that the diameter of an inner staple ejection ring such as 132b or of a staple ejection ring 64 of a mitral valve periphery stapler such as 96 be at least 28 mm in diameter.

Persons suffering from ischemic mitral regurgitation generally have a mitral valve lumen of about 55 mm in diameter. Thus, embodiments of a valve displacement stapler of the present invention, such as stapler 116 having an outer staple ejection ring such as 132a less than 55 mm can easily be treated, where some leaflet area is lost.

One skilled in the art of fastening devices such as staplers is able, without undue experimentation, to implement the teachings of the present invention including manufacturing a stapler of the present invention. For example, in embodiments, anvils of staplers of the present invention such as anvil 66, 100 or 118 are fashioned from a hard material such as known in the field of similar medical devices such as surgical staplers, for example, stainless steel, titanium or alloys thereof. In the embodiments discussed above, stapler 52 and 116 are configured so that a cutting blade 60 and staples 86 contact tissue substantially simultaneously. That said, in non-depicted embodiments, cutting blade 60 contacts tissue prior to staples 86. That said, in embodiments, staples 86 contacts tissue prior to cutting blade 60.

In the embodiments discussed above, a cutting blade 60 is retained in at least a portion of a blade accepting slot 128 so that cutting blade 60 is prevented from falling into the body of a subject (e.g., a left ventricle 28) during the stapling process. That said, in non-depicted embodiments, cutting blade 60 is not actively prevented from falling into the body of a subject.

In the embodiments discussed above, the implants used in implementing the teachings of the present invention, both the patch of tissue used for augmenting a cardiac leaflet and the lumen-defining implant are fashioned substantially from commercially available cross linked bovine pericardium. When implementing the teachings of the present inventions, the implants, whether as patches, or as lumen-defining implants such as sheets with holes, annuli, tubes or other, may comprise any suitable material or combination of materials, whether synthetic or biological. Preferably at least one material from which an implant is fashioned is impermeable to prevent the flow of blood through the implant once implanted.

Typical synthetic materials suitable for fashioning an implant of the present invention include but are not limited to fluorinated hydrocarbons such as polytetrafluoroethylene, urethane, elastomer, polyamide and polyester.

Sources of typical biological materials suitable for fashioning an implant of the present invention include but are not limited to materials from a human source, an equine source, a porcine source or a bovine source. In embodiments, biological materials used for fashioning an implant of the present invention include but are not limited to autologous tissue, homologous tissue and heterologous tissue. Specific examples include venous tissue, arterial tissue, serous tissue, dura mater, pleura, peritoneum, pericardium and aortic leaflet.

In the embodiments discussed above, the lockable fasteners, staples 86, are substantially of stainless steel. In non-depicted embodiments, lockable fasteners are fashioned from a material other than stainless steel that is suitable for implantation in the body. Suitable materials from which to fashion lockable fasteners used in implementing the teachings of the present invention include metals such as stainless steel, silver, silver alloys and titanium alloys as well as non-metal, plastic and polymer materials, see for example, U.S. Pat. No. 5,324,307 and references cited therein.

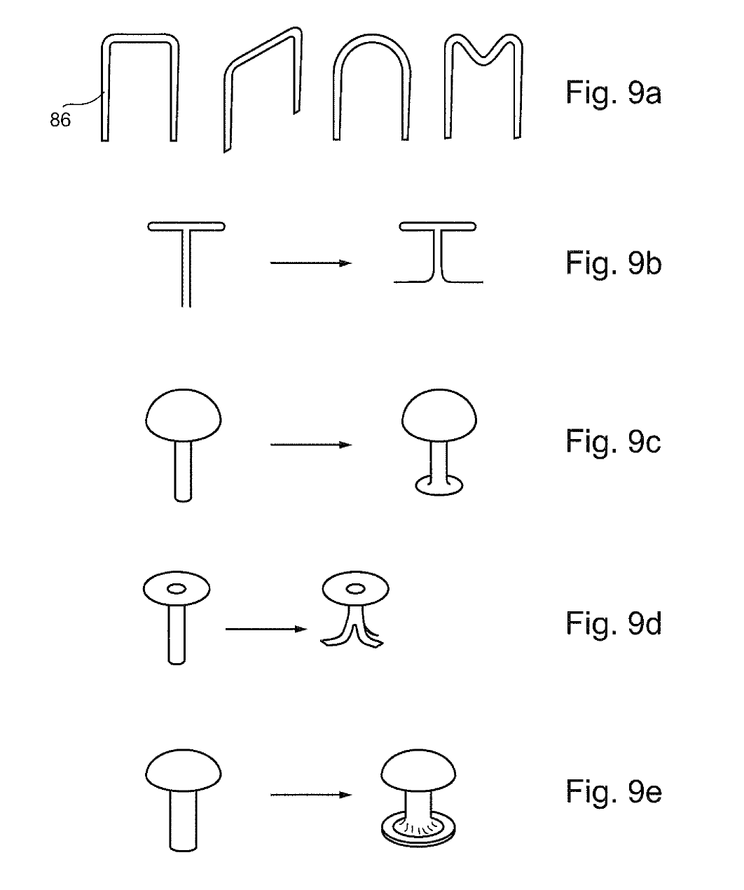

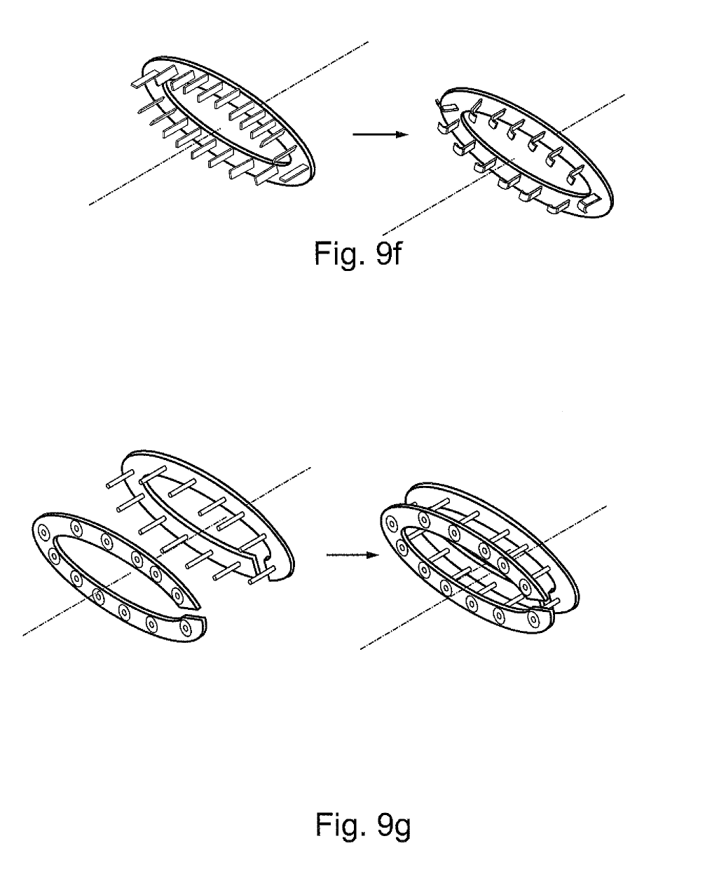

In the embodiments discussed above, the lockable fasteners, staples 86, are substantially of a square U-shape. In embodiments, staples 86 are of a different shape, for example of a skewed square U-shape, a rounded U-shape or an M-shape (FIG. 9A).

In the embodiments discussed above, the lockable fasteners used to implement the teachings of the present invention are staples having two functionally associated legs that penetrate tissue where locking is achieved by deforming the staple, that is bending together of the legs to clamp layers of tissue together. In embodiments, alternative types of lockable fasteners are used in implementing the teachings of the present invention. Generally, lockable fasteners useful in implementing the teachings of the present invention, when implanted, have no part that extends beyond, extends over or encircles an edge of treated membranes but rather pass through the membranes. Generally, lockable fasteners useful in implementing the augmentation of the tissue surrounding a cardiac valve, when implanted, have no part that extends beyond, extends over or passes through a valve lumen so as to avoid substantial interference with leaflet functioning.

In embodiments, alternative lockable fasteners are, like staples, configured to lock by deformation and include, but are not limited to, fasteners such as cotter pins (e.g., FIG. 9B), studs (e.g., FIG. 9C) and rivets (e.g., FIG. 9D). One skilled in the art is able, upon perusal of the specification, to modify the teachings of the present invention to fashion a fastening device that uses an alternative lockable fastener, such as the listed above, including with the appropriately configured anvil to deform the fastener.

In embodiments, alternative lockable fasteners are configured to lock by engaging a discrete locking component such as retaining rings, constricting rings and unidirectional locking rings and include, but are not limited to two-part rivets (e.g., FIG. 9E). One skilled in the art is able, upon perusal of the specification, to modify the teachings of the present invention to fashion a fastening device that uses an alternative fastener that locks with the help of a locking component, such as the listed above. In such embodiments, instead of, or together with, an anvil that acts as a counterpoise to deform a fastener, an analogous component is used which functions as a “tray” to align and orient locking components to allow coupling of a fastener with a respective locking component.

In the embodiments discussed above, a fastening device such as a stapler is configured to implant a plurality of discrete lockable fasteners to define a closed curve. In embodiments, a fastening device is configured to implant a single lockable fastener defining a closed curve, where the single fastener is provided with three or more piercing members. In such embodiments, a fastener comprises a) a loop-shaped body with a bottom side defining a lumen and describing at least a 320° sector about an axis; and b) at least three (in embodiments, four, five, six and even more, e.g., 10 or 20) of elongated piercing elements protruding from the bottom side substantially parallel to the axis.

In embodiments, the loop-shaped body describes at least a 340° sector about the axis and even a complete loop about the axis.

In embodiments, the fastener is lockable by deformation of the (preferably by bending, preferably in a direction tangential to the body) upon sufficiently forceful contact with an anvil when projected from an appropriately configured fastening device so as to lock in place in a manner analogous to a staple, see for example, the lockable fastener depicted in FIG. 9F comprising a loop-shaped body describing a complete closed curve (a circle).

In embodiments, the fastener is lockable by securing at least one discrete locking component to the piercing elements. For example, the lockable fastener depicted in FIG. 90 comprises a loop-shaped body describing a 3400 sector of a closed curve (a circle) and is configured to be locked by passing the piercing elements through the holes provided with pawls to allow only unidirectional passage of the piercing elements) in a one-piece locking ring (split to allow maneuvering around the chordae) which is discrete from the fastener body.

For treating human subjects suffering, for example, from mitral insufficiency, in accordance with the teachings of the present invention it is preferred that the lumen of such a lockable fastener have a diameter of at least about 25 mm and even at least about 28 mm to allow for sufficient flow of blood therethrough. For treating human subjects suffering, for example, from mitral insufficiency, in accordance with the teachings of the present invention, it is preferred that the loop-shaped body have an outer diameter of not more than about 60 mm, and even not more than 55 mm, to allow implantation in a mitral valve annulus without discomfort or damage.

In embodiments, the loop-shaped body and/or the piercing elements are between about 0.1 mm and about 2 mm broad in a radial direction from the axis, which are typical dimensions for surgical staples.

In an embodiment exceptionally useful for implementing augmentation of tissue surrounding a cardiac valve with the help of embodiments of devices of the present invention similar to stapler 116, a set of at least two lockable fasteners such as above is provided, where the two fasteners are coaxially nestable, so that when coaxially nested, a coaxial gap between the outer edge of the body of the smaller fastener and the inner edge of the body of the larger fastener is at least 0.1 mm and preferably less than about 10 mm. Such a coaxial gap is configured to accommodate an implant and a cutting blade, as described above with reference to stapler 116.

The various embodiments of the present invention, especially the methods of augmenting tissue, have been described herein primarily with reference to treatment of living human subjects. It is understood, however, that embodiments of the present invention are performed for the veterinary treatment of a non-human mammal, especially horses, cats, dogs, cows and pigs.

The various embodiments of the present invention, especially the methods of augmenting tissue, have been described herein primarily with reference to treatment of living subjects. It is understood that application of the present invention for training and educational purposes (as opposed to treating a condition) falls within the scope of the claims, whether on a living non-human subject or on a dead subject, whether on a human cadaver or on a non-human body, whether on an isolated cardiac valve, or on a valve in a heart isolated (at least partially) from a body, or on a body.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A surgical fastening device useful for securing a cardiac valve to an implant, comprising:
a) a device body comprising a fastener magazine having a fastener ejection ring configured to project a plurality of tissue-piercing elements of one or more fasteners in the shape of a closed curve from said fastener magazine; and
b) an anvil configured to act as a counterpoise during said projection of said tissue-piercing elements which comprises an anvil head including an anvil head top surface having a shape of a closed-curve and a size substantially similar to that of said fastener ejection ring, said anvil configured to be reversibly secured to said device body through a stem surrounded by said fastener ejection ring, so that when secured, said anvil head surface is opposite said fastener ejection ring
wherein said anvil head comprises at least two mateable anvil head parts, wherein each said mateable anvil head part spans a sector of said closed curve of said anvil head.

2. The device of claim 1, wherein said one or more fasteners are selected from the group consisting of staples, cotter pins, studs, rivets, and two-part rivets.

3. An anvil for a surgical fastening device useful for securing a cardiac valve to an implant, comprising at least two mateable anvil head parts that when mated said anvil head parts constitute an anvil head that is substantially a closed curve including an anvil head top surface, wherein each said mateable anvil head part spans a sector of said closed curve, and a width of said mateable anvil head part is less than half of a radius of said closed curve.

4. The anvil of claim 3, comprising two mateable anvil head parts, a first anvil head part and a second anvil head part, when mated together spanning the entire circumference of said closed curve.

5. The anvil of claim 4, wherein said first anvil head part spans a sector of between about 120° and about 240° of said closed curve.

6. The anvil of claim 3, each said anvil head part comprising crosspiece extending toward a center of said closed curve.

7. The anvil of claim 6, a said crosspiece spanning no greater than about 45°.

8. The anvil of claim 3, further comprising a stem attached to at least one said anvil head part through a crosspiece.

9. The anvil of claim 8, said stem attached to only one said anvil head part.

10. The anvil of claim 8, said stem split into a plurality of mateable parts, said parts attached to different said anvil head parts, configured so that when said anvil head parts are properly mated said stem parts are properly mated.

11. The anvil head of claim 3, said top surface comprising a tray for presenting locking components to engage fasteners projected from the surgical fastening device.

12. The anvil head of claim 3, said top surface comprising features for facilitating deformation of fasteners projected from the surgical fastening device.

13-37. (canceled)

38. A lockable fastener useful for securing the periphery of a cardiac valve to an implant material comprising:
a) a loop-shaped body with a bottom side defining a lumen and describing at least a 320° sector about an axis; and
b) at least three elongated piercing elements protruding from said bottom side substantially parallel to said axis.

39. The fastener of claim 38, said piercing elements configured to deform upon sufficiently forceful contact with an anvil when projected from an appropriately configured surgical fastening device so as to lock in place.

40. The fastener of claim 38, a said piercing element configured to engage a discrete locking component.

41. A set of lockable fasteners, comprising at least two fasteners of claim 38 configured to be coaxially nestable, so that when coaxially nested, a coaxial gap between an outer edge of a said loop-shaped body of a smaller said fastener and an inner edge of a said loop-shaped body of a larger said fastener is at least 0.1 mm.

42. A method of augmenting a membrane, comprising:
a) slitting an in vivo membrane so as to produce a slit having a first slit edge and a second slit edge;
b) providing a patch of tissue having a periphery as a membrane augmenting implant;
c) placing said patch of tissue to define an overlap region comprising an overlap of a first portion of said periphery with said first slit edge and an overlap of a second portion of said periphery with said second slit edge; and
d) securing said patch to said membrane around a plurality of locations of said overlap region substantially simultaneously,
thereby augmenting said membrane by increasing a surface area of said membrane with said patch.

43-46. (canceled)

47. The method of claim 42, wherein said securing comprises:
i) piercing said patch and said membrane at said plurality of locations to make a plurality of holes in said overlap region substantially simultaneously;
ii) passing lockable fasteners through said holes; and
iii) locking said lockable fasteners so as to secure said patch to said first slit edge and said second slit edge through said overlap region.

48. The method of claim 42, wherein said in vivo membrane is a leaflet of a cardiac valve.

49. A method of augmenting the tissue surrounding a cardiac valve, comprising:
a) excising leaflets of a cardiac valve with an incision having a shape of a closed curve so as to define a valve seat edge of said incision and a valve periphery edge of said incision;
b) providing an implant including a wall, the wall delimited by two edges each in the shape of a closed curve and defining a lumen as a cardiac valve augmenting implant;
c) placing said implant to define a proximal overlap region comprising an overlap of a first portion near a first edge of said implant with said valve seat edge
d) placing said implant to define a distal overlap region comprising an overlap of a second portion near a second edge of said implant with said valve periphery edge;
e) securing said first portion of said implant to said valve seat edge around a plurality of locations of said proximal overlap region; and
f) securing said second portion of said implant to said valve periphery edge around a plurality of locations of said distal overlap region, substantially simultaneously thereby augmenting a surface area of tissue surrounding said cardiac valve with said implant.

50. The method of claim 49, wherein said securing said first portion of said implant to said valve seat edge around a plurality of locations of said proximal overlap region is performed substantially simultaneously for said plurality of locations.

51. The method of claim 49, wherein:
said excising;
said placing said implant to define said proximal overlap zone; and
said securing said first portion of said implant to said valve seat edge are substantially simultaneous.

52. The method of claim 49, wherein:
said excising;
said placing said implant to define said distal overlap zone; and
said securing said second portion of said implant to said valve periphery edge are substantially simultaneous.

53. The method of claim 49, wherein:
said excising;
said placing said implant to define said proximal overlap zone;
said placing said implant to define said distal overlap zone;
said securing said first portion of said implant to said valve seat edge; and
said securing said second portion of said implant to said valve periphery edge are substantially simultaneous.

54-56. (canceled)

57. The method of claim 49, wherein said securing said first portion of said implant to said valve seat edge comprises:
i) piercing said implant and tissue surrounding said valve seat edge at said plurality of locations to make a plurality of holes in said proximal overlap region substantially simultaneously;
ii) passing lockable fasteners through said holes; and
iii) locking said lockable fasteners so as to secure said implant to said valve seat edge through said proximal overlap region.

58. The method of claim 49, wherein securing said second portion of said implant to said valve periphery edge comprises:
i) piercing said implant and tissue surrounded said valve periphery edge at said plurality of locations to make a plurality of holes in said distal overlap region substantially simultaneously;
ii) passing lockable fasteners through said holes; and
iii) locking said lockable fasteners so as to secure said implant to said valve periphery edge through said distal overlap region.

59. The method of claim 49, wherein said cardiac valve is a mitral valve.

60. The anvil of claim 3, configured so that at least one of the anvil head parts is shaped to pass between the cardiac valve chord and a ventricle wall.

61. The anvil of claim 60, configured so that when mated said closed curve is sized to go around the chordae of the cardiac valve.

62. The anvil of claim 3, configured so that at least one of the anvil head parts is shaped to pass between the cardiac valve chord and a ventricle septum.

63. The anvil of claim 62, configured so that when mated said closed curve is sized to go around the chordae of the cardiac valve.
